(19) United States
(12) Patent Application Publication
(10) Pub. No.: US 2006/0116713 A1
(43) Pub. Date: Jun. 1, 2006

(54) Aneurysm treatment devices and methods

(76) Inventors: Ivan Sepetka, Los Altos, CA (US); Maria Aboytes, Palo Alto, CA (US); Maybelle Jordan, Potomac, MD (US); Craig Friedman, Westport, CT (US); Arindam Datta, Hillsboro, NJ (US)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/229,044

(22) Filed: Sep. 15, 2005

Related U.S. Application Data
(63) Continuation-in-part of application No. 11/111,487, filed on Apr. 21, 2005.
Application No. 11/111,487 is a continuation-in-part of application No. 10/998,357, filed on Nov. 26, 2004.

(51) Int. Cl. A61M 29/00

(52) U.S. Cl. 606/200

(57) ABSTRACT

An aneurysm treatment device for in situ treatment of aneurysms comprises an occlusion device having a flexible, longitudinally extending elastomeric matrix member that assumes a non-linear shape to conformally fill a targeted vascular site. The occlusion device has one or more longitudinally extending filaments that can be varied to impart properties to the occlusion device.

### CROSS-REFERENCE TO RELATED APPLICATIONS

This application is a continuation-in-part of co-pending, commonly assigned Ser. No. 11/111,487, filed Apr. 21, 2005, which in turn is a continuation-in-part of co-pending, commonly assigned U.S. patent application Ser. No. 10/998,357, filed Nov. 26, 2004, both of which are is incorporated herein by reference.

### FIELD OF THE INVENTION

This invention relates to methods and devices for the treatment of vascular aneurysms and other comparable vascular abnormalities. More particularly, this invention relates to occlusion devices for vascular aneurysms that comprise a reticulated elastomeric matrix structure and a delivery device.

### BACKGROUND OF THE INVENTION

The cardiovascular system, when functioning properly, supplies nutrients to all parts of the body and carries waste products away from these parts for elimination. It is essentially a closed system comprising the heart, a pump that supplies pressure to move blood through the blood vessels, blood vessels that lead away from the heart, called arteries, and blood vessels that return blood toward the heart, called veins. On the discharge side of the heart is a large blood vessel called the aorta from which branch many arteries leading to all parts of the body, including the organs. As the arteries get close to the areas they serve, they diminish to small arteries, still smaller arteries called arterioles, and ultimately connect to capillaries. Capillaries are minute vessels where outward diffusion of nutrients, including oxygen, and inward diffusion of wastes, including carbon dioxide, takes place.

Capillaries connect to tiny veins called venules. Venules in turn connect to larger veins which return the blood to the heart by way of a pair of large blood vessels called the inferior and superior venae cava.

When the wall 2 of an artery 4 has a weakness, the blood pressure can dilate or expand the region of the artery 4 with the weakness, and a pulsating sac 6 called a berry or saccular aneurysm (FIG. 1), can develop. Saccular aneurysms are common at artery bifurcations 8 (FIGS. 2 and 3) located around the brain. Dissecting aneurysms are common in the thoracic and abdominal aortas. The pressure of an aneurysm against surrounding tissues, especially the pulsations, may cause pain and may also cause tissue damage. However, aneurysms are often asymptomatic. The blood in the vicinity of the aneurysm can become turbulent, leading to formation of blood clots, that may be carried to various body organs where they may cause damage in varying degrees, including cerebrovascular incidents, myocardial infarctions and pulmonary embolisms. Should an aneurysm tear and begin to leak blood, the condition can become life threatening, sometimes being quickly fatal, in a matter of minutes.

Because there is relatively little blood pressure in a vein, venous “aneurysms” are non-existent. Therefore, the description of the present invention is related to arteries, but applications within a vein, if useful, are to be understood to be within the scope of this invention.

The causes of aneurysms are still under investigation. However, researchers have identified a gene associated with a weakness in the connective tissue of blood vessels that can lead to an aneurysm. Additional risk factors associated with aneurysms such as hyperlipidemia, atherosclerosis, fatty diet, elevated blood pressure, smoking, trauma, certain infections, certain genetic disorders, such as Marfan's Syndrome, obesity, and lack of exercise have also been identified. Cerebral aneurysms frequently occur in otherwise healthy and relatively youthful people and have been associated with many untimely deaths.

Aneurysms, widening of arteries caused by blood pressure acting on a weakened arterial wall, have occurred ever since humans walked the planet. In recent times, many methods have been proposed to treat aneurysms. For example, Greene, Jr., et al., U.S. Pat. No. 6,165,193 proposes a vascular implant formed of a compressible foam hydrogel that has a compressed configuration from which it is expansible into a configuration substantially conforming to the shape and size of a vascular malformation to be embolized. The hydrogel of the '193 patent lacks the mechanical properties to enable the hydrogel to regain its size and shape in vivo were it to be compressed for catheter, endoscope, or syringe delivery, and the process can be complex and difficult to implement. Other patents disclose introduction of a device, such as a stent or balloon (Naglreiter et al., U.S. Pat. No. 6,379,329) into the aneurysm, followed by introduction of a hydrogel in the area of the stent to attempt to repair the defect (Sawhney et al., U.S. Pat. No. 6,379,373).

Ferrera et al., U.S. Published Patent Application No. 2003/0199887 discloses that a porous or textural embolization device comprising a resilient material can be delivered to a situs of a vascular dysfunction. The device has a relaxed state and a stretcned state, where the relaxed state forms a predetermined space-filling body.

Still other patents suggest the introduction into the aneurysm of a device, such as a stent, having a coating of a drug or other bioactive material (Gregory, U.S. Pat. No. 6,372,228). Other methods include attempting to repair an aneurysm by introducing via a catheter a self-hardening or self-curing material into the aneurysm. Once the material cures or polymerizes in situ into a foam plug, the vessel can be recanalized by placing a lumen through the plug (Hastings, U.S. Pat. No. 5,725,568).

Another group of patents relates more specifically to saccular aneurysms and teaches the introduction of a device, such as string, wire or coiled material (Boock, U.S. Pat. No. 6,312,421), or a braided bag of fibers (Greenhalgh, U.S. Pat. No. 6,346,117) into the lumen of the aneurysm to fill the void within the aneurysm. The device introduced can carry hydrogel, drugs, or other bioactive materials to stabilize or reinforce the aneurysm (Greene Jr., et al., U.S. Pat. No. 6,299,619).

Another treatment known to the art comprises catheter delivery of platinum microcoils into the aneurysm cavity in conjunction with an embolizing composition comprising a biocompatible polymer and a biocompatible solvent. The deposited coils or other non-particulate agents are said to act as a lattice about which a polymer precipitate grows thereby embolizing the blood vessel (Evans et al., U.S. Pat. No. 6,335,384).

It is an understanding of the present invention that such methods and devices suffer from a variety of problems. For example, if an aneurysm treatment is to be successful, any implanted device must be present in the body for a long period of time, and must therefore be resistant to rejection and not degrade into materials that cause adverse side effects. While platinum coils may have some benefits in this respect, they are inherently expensive, and the pulsation of blood around the aneurysm may cause difficulties such as migration of the coils, incomplete sealing of the aneurysm, or fragmentation of blood clots. It is also well known that the use of a coil is frequently associated with recanalization of the site, leading to full or partial reversal of the occlusion. If the implant does not fully occlude the aneurysm and effectively seal against the aneurysm wall, pulsating blood may seep around the implant and the distended blood vessel wall causing the aneurysm to reform around the implant.

The delivery mechanics of many of the known aneurysm treatment methods can be difficult, challenging, and time consuming.

Most contemporary vascular occlusion devices, such as coils, thrombin, glue, hydrogels, etc., have serious limitations or drawbacks, including, but not limited to, early or late recanalization, incorrect placement or positioning, migration, and lack of tissue ingrowth and biological integration. Also, some of the devices are physiologically unacceptable and engender unacceptable foreign body reactions or rejection. In light of the drawbacks of the known devices and methods, there is a need for more effective aneurysm treatment that produces permanent biological occlusion, can be delivered in a compressed state through small diameter catheters to a target vascular or other site with minimal risk of migration, and/or will prevent the aneurysm from leaking or reforming.

### OBJECTS OF THE INVENTION

It is an object of the invention to provide a method and device for the treatment of vascular aneurysms.

It is also an object of the invention to provide a method and device for occluding cerebral aneurysms.

It is a further object of the invention to provide a method and device for occluding cerebral aneurysms by bio-integrating and sealing off the aneurysm to prevent migration, recanalization, leaking, or reforming.

It is a yet further object of the invention to provide a method and device for occluding vascular aneurysms wherein the device comprises a biocompatible member and a delivery device.

It is a yet further object of the invention to provide a method and device for occluding vascular aneurysms comprising a biocompatible member and two or more longtitudinally extending components.

It is a yet further object of the invention to provide a system for treating cerebral aneurysms that comprises a reticulated elastomeric matrix structure and a delivery device.

It is a yet further object of the invention to provide an occlusion device comprising a flexible, longitudinally extending elastomeric matrix member, wherein the device assumes a non-linear shape to conformably fill a targeted vascular site.

It is a yet further object of the invention to provide an occlusion device comprising an elastomeric matrix and one or more structural filaments.

It is a yet further object of the invention to provide an occlusion device wherein the structural components comprise platinum wire and polymeric fiber or filament.

It is a yet further object of the invention to provide a method of preparing an occlusion device comprising an elastomeric matrix and one or more structural filaments.

It is a yet further object of the invention to provide a method of occluding a vascular aneurysm wherein an occlusion device comprising an elastomeric matrix and one or more structural filaments conformally fills a targeted vascular site.

These and other objects of the invention will become more apparent in the discussion below.

### SUMMARY OF THE INVENTION

According to the invention an aneurysm treatment device is provided for in situ treatment of aneurysms, particularly, cerebral aneurysms, in mammals, especially humans. The treatment device comprises a resiliently implant comprised of a reticulated, biodurable elastomeric matrix and one or more structural filaments, wherein the implant is deliverable into the aneurysm, for example, by being loadable into a catheter and passed through a patient's vasculature. Pursuant to the invention, useful aneurysm treatment devices can have sufficient resilience, or other mechanical properties, including expansion, to conformally fill the space within the aneurysm sac and to occlude the aneurysm.

In another embodiment of the invention, an implant comprises one or more flexible, connected, preferably spherically-, ellipsoidally-, or cylindrically-shaped structures that are positioned in a stretched state in a delivery catheter. The connected structures preferably have spring coils on each end, one of which coils is releasably secured within the delivery catheter.

In another embodiment of the invention, an implant for occlusion of an aneurysm comprises reticulated elastomeric matrix in a shape that can be inserted into a delivery catheter, can be ejected or deployed from the delivery catheter into an aneurysm, and can then be of sufficient size and shape to conformally fill and occlude the aneurysm. Examples of such shapes include, but are not limited to, cylinders, hollow cylinders, noodles, hollow cylinders with lateral slots, rods, tubes, or elongated prismatic forms, coiled, helical or other more compact configurations, segmented cylinders where “sausage-like” segments have been formed, braided shapes, or flat spiral shapes, optionally with one or more structural filaments such as polymeric fiber or filament or radiopaque wire support extending therein.

In another embodiment of the invention, an aneurysm occlusion device comprises elastomeric matrix in the nature of a string or other elongate form and having one or more structural filaments. Preferably the filaments comprise one or more platinum wires and polymeric fiber or filament. The occlusion device may optionally have lateral components to impart chain-like behavior when the occlusion device is advanced to conformally fill an aneurysm sac or cavity.

Although multiple implants can be deployed, used, or implanted, it is a feature of one aspect of the present invention that preferably a single implant fills an aneurysm, effectively a “single shot” occlusion. It is contemplated, in one embodiment, that even when their pores become partially filled or completely filled with biological fluids, bodily fluids and/or tissue in the course of time or immediately after delivery, and/or the implants are either still partially compressed or partially recovered after delivery, such implantable device or devices for vascular malformation applications have a volume, prior to packing in vivo, of at least about 50% of the aneurysm volume. The ratio of implant (or implants) volume to aneurysm volume is defined as packing density. In another embodiment, such implantable device or devices for vascular malformation applications have a volume, prior to packing in vivo, of at least about 75% of the aneurysm volume. In another embodiment, such implantable device or devices for vascular malformation applications have a volume, prior to packing in vivo, of at least about 125% of the aneurysm volume. In another embodiment, such implantable device or devices for vascular malformation applications have a volume, prior to packing in vivo, of at least about 175% of the aneurysm volume. In another embodiment, such implantable device or devices for vascular malformation applications have a volume, prior to packing in vivo, of at least about 200% of the aneurysm volume.

The packing density is targeted to achieve angiographic occlusion after embolization of the aneurysm by the implant, followed by clotting, thrombosis, and tissue ingrowth, ultimately leading to biological obliteration of the aneurysm sac. Permanent tissue ingrowth will prevent any possible recanalization or migration.

It is furthermore preferable that the implant be treated or formed of a material that will encourage such fibroblast immigration. It is also desirable that the implant be configured, with regard to its three-dimensional shape, and its size, resiliency and other physical characteristics, and be suitably chemically or biochemically constituted to foster eventual tissue ingrowth and formation of scar tissue that will help conformally fill the aneurysm sac.

The aneurysm treatment according to the invention device comprises, in one embodiment, a reticulated biodurable elastomeric matrix or the like that is capable of being inserted into a catheter for implantation. In another embodiment, the implant can be formed of a partially hydrophobic reticulated biodurable elastomeric matrix having its pore surfaces coated to be partially hydrophilic, for example, by being coated with at least a partially hydrophilic material, optionally a partially hydrophilic reticulated elastomeric matrix. The entire elastomeric matrix may have such a hydrophilic coating throughout the pores of the reticulated elastomeric matrix.

In one embodiment, the hydrophilic material carries a pharmacologic agent, for example, elastin or fibrin to foster fibroblast proliferation. It is also within the scope of the invention for the pharmacologic agent to include sclerotic agents, inflammatory induction agents, growth factors capable of fostering fibroblast proliferation, or genetically engineered an/or genetically acting therapeutics. The pharmacologic agent or agents preferably are dispensed over time by the implant. Incorporation of biologically active agents in the hydrophilic phase of a composite foam suitable for use in the practice of the present invention is described in co-pending, commonly assigned U.S. patent applications Ser. No. 10/692,055, filed Oct. 22, 2003, Ser. No. 10/749,742, filed Dec. 30, 2003 (published Feb. 24, 2005 as U.S. Patent Publication No. 20050043585), Ser. No. 10/848,624, filed May 17, 2004 (published Feb. 24, 2005 as U.S. Patent Publication No. 20050043816), and Ser. No. 10/900,982, filed Jul. 27, 2004, each of which is incorporated herein by reference in its entirety.

In another aspect, the invention provides a method of treating an aneurysm comprising the steps of:
imaging an aneurysm to be treated to determine its size and topography;
selecting an aneurysm treatment device according to the invention for use in treating the aneurysm; and
implanting the aneurysm treatment device into the aneurysm.

Preferably, the method further comprises:
loading the aneurysm treatment device into a catheter or other delivery means;
threading the catheter through an artery to the aneurysm; and
positioning and releasing the aneurysm treatment device in the aneurysm.

Once an aneurysm has been identified using suitable imaging technology, such as a magnetic resonance image (MRI), computerized tomography scan (CT Scan), x-ray imaging with contrast material or ultrasound, and is to be treated, the surgeon chooses which implant he or she feels would best suit the aneurysm, both in shape and size. The implant can be used alone. In another embodiment, the aneurysm treatment device of the invention may also be used in conjunction with a frame of platinum coils or with a stent or balloon across the neck of the aneurysm, to assist in reducing or eliminating the risk of implant migration out of the neck of the aneurysm. This is particularly true in the case of wide neck or giant aneurysms. The chosen implant is then loaded into an intravascular catheter in a linear state. If desired, the implant can be provided in a sterile package in a pre-loaded configuration, ready for loading into a catheter. Alternatively, the implants can be made available in an expanded state, also, preferably, in a sterile package, and the surgeon at the site of implantation can use a suitable secondary device or a loader apparatus to compress an implant so that it can be loaded into a delivery catheter.

With an implant loaded into the catheter, the catheter is advanced through an artery to the diseased portion of the affected artery using any suitable technique known in the art. By use of the catheter the implant is then inserted and positioned within the aneurysm. As the implant is released from the catheter, where it is manipulated into a suitable position within the aneurysm.

In another embodiment of the invention, a vascular occlusion device comprises:

a flexible, longitudinally extending biocompatible member, and

at least one longitudinally extending component coupled to the biocompatible member at various points to secure the biocompatible member and assist it in conformally filling a targeted vascular site.

In another embodiment of the invention, the device assumes a non-linear shape to conformally fill a targeted vascular site.

In another embodiment of the invention, the device comprises a non-curvilinear shape in at least one portion of the member.

In another embodiment of a device of the invention, the non-curvilinear shape comprises at least one vertex.

In another embodiment of a device of the invention, the at least one vertex comprises a plurality of vertices.

In another embodiment of a device of the invention, the plurality of vertices permit chain-like folding of the device.

In another embodiment of a device of the invention, the biocompatible member comprises an elastomeric matrix.

In another embodiment of a device of the invention, the elastomeric matrix is a biodurable, reticulated elastomeric matrix.

In another embodiment of a device of the invention, the elastomeric matrix is selected from the group consisting of polycarbonate polyurethane-urea, polycarbonate polyurea-urethane, polycarbonate polyurethane, and polycarbonate polysiloxane polyurethane.

In another embodiment of a device of the invention, the elastomeric matrix comprises resiliently recoverable material.

In another embodiment of a device of the invention, each longitudinally extending component comprises a structural filament.

In another embodiment of a device of the invention, the at least one longitudinally extending components comprise a polymeric fiber or filament and at least one wire element.

In another embodiment of a device of the invention, the at least one wire element comprises a continuous wire.

In another embodiment of a device of the invention, the at least one wire element comprises a plurality of staples, preferably interlocked to form a chain.

In another embodiment of the invention, the device comprises at least two longitudinally extending components that are coupled to each other at a plurality of locations.

In another embodiment of a device of the invention, the components are coupled by knotting.

In another embodiment of a device of the invention, the at least one longitudinally extending components comprise at least two structural filaments.

In another embodiment of a device of the invention, there are two structural filaments.

In another embodiment of a device of the invention, the structural filaments are selected from materials preselected to vary at least one physical property of the device.

In another embodiment of a device of the invention, the physical property is stiffness.

In another embodiment of a device of the invention, the physical property comprises modulus of elasticity.

In another embodiment of a device of the invention, each structural filament is selected from the group consisting of platinum wire, polymeric fiber or filament, a braid of platinum wire and polymeric fiber or filament, and a braid of two or more platinum wires.

In another embodiment of a device of the invention, the structural filaments are knotted together by radiopaque bands.

In another embodiment of a device of the invention, at least one longitudinally extending component is radiopaque.

In another embodiment of a device of the invention, the material of each component and the coupling between the at least two components are selected to produce a desired physical property of the device.

In another embodiment of the invention, the desired physical property of the device comprises stiffness at a location of coupling and the stiffness comprises a stiffness relative to a stiffness of the device at a point substantially distant from the point of coupling.

In another embodiment of the invention, the stiffness as measured at the point of coupling is measured relative to a stiffness of the device at a point substantially distant from the point of coupling.

In another embodiment of the invention, the device is capable of occluding an aneurysm, such as a cerebral aneurysm.

In another embodiment of the invention, the device is capable of occluding a vessel or vascular malformation.

In another embodiment of the invention, an introducer system for a vascular occlusion device, the vascular occlusion device having a proximal end and a distal end, the distal end having a contact element coupled to it, comprises:

an introducer component having a longitudinally extending lumen and proximal and distal ends;

a pusher component slidable within the introducer component, the pusher component having a distal end positioned adjacent to the distal end of the occlusion device; and

a core component having a distal end and extending through the pusher component and parallel to the occlusion device so that the distal end of the core component contacts the contact element, thereby applying a tensile force to the occlusion device.

In another embodiment of the invention, an introducer system further comprises:

an interlocking wire having a distal end extending longitudinally into the pusher member,

wherein:

the occlusion device has a release element at its proximal end,

the distal end of the pusher component has an opening through which the release element extends,

the distal end of the interlocking wire is releasably held within the distal end of the pusher member, and

the distal end of the interlocking wire releasably engages the release element so that the distal end of the pusher component releasably engages the proximal end of the occlusion device.

In another embodiment of an introducer system of the invention, the release element comprises a loop.

In another embodiment of an introducer system of the invention, the contact element is a tensioning element.

In another embodiment of the invention, a method for occluding a targeted vascular site comprises:

introducing an introducer system into a delivery catheter having a longitudinally extending lumen and proximal and distal ends, the introducer system carrying a vascular occlusion device and having a pusher component;

withdrawing the introducer system, leaving the vascular occlusion device positioned within the lumen of the delivery catheter;

advancing the vascular occlusion device using the pusher component to position the vascular occlusion device within the targeted vascular site;

disengaging the pusher component from the occlusion device; and

withdrawing the pusher.

In another embodiment of the invention, a device for occluding a targeted vascular site comprises:

an elongate occluding element comprising a material permitting ingrowth of tissue at the targeted vascular site; and

a plurality of features provided along the occluding element at preselected locations, the features selected to confer material characteristics allowing the creation of vertices in the element.

In another embodiment of a device of the invention, the vertices are at least temporary.

In another embodiment of a device of the invention, the vertices facilitate packing of the occluding element into the targeted vascular site.

In another embodiment of a device of the invention, at least one of the features comprises a topological characteristic of the elongate element.

In another embodiment of the invention, the device further comprises a second element coupled to the elongate element, wherein at least one of the features comprises topological characteristic of the second element.

In another embodiment of a device of the invention, the device further comprises a third element coupled to the elongate element, wherein at least one of the features comprises a relationship between the second and third elements.

In another embodiment of a device of the invention, the elongate element comprises a biodurable material permitting vascular tissue ingrowth and the second element comprises a polymeric fiber or filament.

In another embodiment of a device of the invention, the topological characteristic of the polymeric fiber or filament comprises a stitch.

In another embodiment of a device of the invention, the relationship between the second and third elements comprises a knot.

In another embodiment of a device of the invention, at least one of the group consisting of a dimension of a feature and a distance between a pair of features is preselected to facilitate packing of the targeted vascular site.

In another embodiment of the invention, a method for treating a condition at a targeted vascular site comprises the steps of:

providing an elongate occlusion device comprising biocompatible material;

introducing the occlusion device into the targeted vascular site; and

while introducing the occlusion device, inducing at least one non-curvilinear geometry in the occlusion device.

In another embodiment of a method of the invention, the step of inducing at least one non-curvilinear geometry produces a geometry of the occlusion device that packs the targeted vascular site in a substantially conformal manner.

In another embodiment of a method of the invention, the at least one non-curvilinear geometry comprises a plurality of folds.

In another embodiment of a method of the invention, the step of inducing a plurality of folds produces a chain-like occlusion device for packing the targeted vascular site in a substantially conformal manner.

In another embodiment of a method of the invention, the occlusion device comprises a biocompatible material.

In another embodiment of a method of the invention, the biocompatible material comprises a material permitting ingrowth of tissue at the targeted site.

In another embodiment of a method of the invention, the occlusion device is introduced to permanently biointegrate at the targeted site.

In another embodiment of the invention, a method for treating an aneurysm in a mammal comprises the steps of:

providing an elongate biocompatible, biodurable material permitting tissue ingrowth at the site of the aneurysm; and

introducing the biocompatible, biodurable material at the site of the aneurysm in a quantity sufficient to occlude the aneurysm and to permit permanent biointegration of the occlusion device in the aneurysm.

In another embodiment of the invention, the biocompatible, biodurable material is a reticulated elastomeric matrix.

In another embodiment of the invention, a method for treating a cerebral aneurysm comprises the step of introducing sufficient biocompatible material into the cerebral aneurysm to pack the aneurysm with the material to a packing density of from at least about 10% to at least about 200%.

In another embodiment of a method of the invention, the biocompatible material comprises a flexible, longitudinally extending biocompatible member.

In another embodiment of a method of the invention, the biocompatible material comprises non-swellable material.

In another embodiment of the invention, a mechanism for detaching a vascular implant from a delivery device, the vascular implant having a proximal end and a coupling component at its proximal end, comprises:

an engagement element coupled at a distal end of the delivery device, the engagement element having a first, engaged position and a second, disengaged position; and

an energy transfer component coupled to the engagement element at a distal portion of the component to actuate the engagement element,

wherein the engagement element, when actuated, engages the coupling component of the implant when in the first position and releases the coupling component when in the second position.

In another embodiment of a mechanism of the invention, the coupling component of the implant comprises a flexible structure.

In another embodiment of a mechanism of the invention, the flexible structure comprises at least one opening through which an aspect of the engagement element of the delivery device may pass when in the first, engaged position.

In another embodiment of a mechanism of the invention, the flexible structure comprises a loop.

In another embodiment of a mechanism of the invention, the engagement element comprises a structure that moves, along an axis, from the first position to the second position.

In another embodiment of a mechanism of the invention, the delivery device comprises at least one of the group consisting of a wire and a sheath, the axis is parallel to the longitudinal axis of the delivery device, and the energy transfer component comprises at least one of the wire and the sheath.

In another embodiment of a mechanism of the invention, the delivery device comprises a sheath and the energy transfer component comprises a wire, and the engagement element transitions between the first position and the second position as a result of a relative rotation of the wire engagement element with respect to the delivery device sheath.

In another embodiment of a mechanism of the invention, the engagement element comprises a distal portion of the wire, the coupling component of the implant comprises a loop structure, and, in the first position of the engagement element, the loop structure is stably retained about a distal portion of the wire and, in the second position of the engagement element, the loop structure is released over a free distal end of the wire.

In another embodiment of a mechanism of the invention,

the distal portion of the wire has threads that engage mating threads coupled to the sheath,

the delivery device comprises a distal portion having a side wall with an aperture through which the loop structure passes and is held in place when the engagement element is in the first position, and

when the engagement element is in the second position, the distal end of the wire is proximal of the aperture, releasing the loop structure and allowing it to exit through the aperture.

In another embodiment of a mechanism of the invention, the control element is operable by a practitioner.

In another embodiment of the invention, a method for fabricating a vascular occlusion device comprises the steps of:

providing a biocompatible material adapted for tissue ingrowth and capable of being formed into at least one elongate element having a longitudinal axis and dimensioned for vascular insertion;

coupling at least one support element to the biocompatible material to at least partially lie substantially along at least a portion of the longitudinal axis of the at least one elongate element; and

forming the elongate element from the biocompatible material substantially in the vicinity of the longitudinal axis.

In another embodiment of a method of the invention, the elongate element comprises a flexible linear element.

In another embodiment of a method of the invention, the at least one support element comprises a structural filament coupled to the biocompatible material substantially along at least a portion of its longitudinal axis.

In another embodiment of a method of the invention, the at least one support element comprises polymeric fiber or filament.

In another embodiment of a method of the invention, the polymeric fiber or filament is stitched to the biocompatible material.

In another embodiment of a method of the invention, the polymeric fiber or filament is coupled to the biocompatible material with at least one adhesive.

In another embodiment of a method of the invention, the stitching is performed by a sewing machine.

In another embodiment of a method of the invention, the at least one support element further comprises a second support element.

In another embodiment of a method of the invention, the second support element comprises a staple.

In another embodiment of a method of the invention, the at least one support element comprises at least two staples interlocking with one another to form a chain.

In another embodiment of a method of the invention, the at least one second support element comprises a radiopaque material.

In another embodiment of a method of the invention, the at least one second support element comprises wire.

In another embodiment of a method of the invention, the wire is coupled to the polymeric fiber or filament at a plurality of points.

In another embodiment of a method of the invention, the coupling at at least one of the plurality of points comprises a knot.

In another embodiment of a method of the invention, the at least one support element comprises at least two elements including a braided platinum wire/polymeric fiber or filament filament subassembly and a polymeric fiber or filament element.

In another embodiment of a method of the invention, the at least second support element comprises a plurality of staples.

In another embodiment of a method of the invention the staples are spaced apart from one another.

In another embodiment of a method of the invention, the step of forming the elongate element from the biocompatible material and the coupled support element comprises separating the elongate element and the support element from adjoining material.

In another embodiment of a method of the invention, the step of separating is accomplished by cutting.

In another embodiment of a method of the invention, the method further comprises the step of removing excess material so that the elongate element has a preselected maximum width.

In another embodiment of a method of the invention, the method further comprises the step of coupling a visualizable element proximate to the end of the elongate element.

In another embodiment of a method of the invention, the visualizable end unit comprises a coil.

In another embodiment of a method of the invention, the end unit comprises a radiopaque material.

In another embodiment of a method of the invention, the length of the elongate element is from about 1 mm to about 1500 mm, preferably from about 50 mm to about 250 mm.

In another embodiment of a method of the invention, the width of the elongate member is from about 0.25 mm to about 12 mm, preferably from about 0.25 mm to about 0.5 mm.

In another embodiment of a method of the invention, the biocompatible material comprises an elastomeric matrix sheet material having a thickness of from about 1 mm to about 2 mm.

In another embodiment of a method of the invention, the stitching of the suture to the biocompatible material forms a continuous stitch line from about 100 mm to about 500 mm long.

In another embodiment of a method of the invention, the step of coupling at least one support element to the biocompatible material precedes the step of forming the elongate element from the biocompatible material, whereby the elongate element so formed includes the at least one support element.

In another embodiment of a method of the invention, the step of forming the elongate element from the biocompatible material precedes the step of coupling at least one support element to the biocompatible material.

In another embodiment of the invention, a method for treating an aneurysm comprises the steps of:

providing a biocompatible element having a form having at least one portion that lacks a predefined geometry; and

introducing the biocompatible element to conformally fill the aneurysm.

In another embodiment of a method of the invention, the step of introducing the biocompatible material comprises application of the material to a wall of the aneurysm in such a manner that material curves upon itself to produce segments of the material.

In another embodiment of a method of the invention, the material segments so applied are arranged in a brush stroke form.

In another embodiment of a method of the invention, the segments, although substantially parallel to the wall of the aneurysm, each have a spatial orientation, and the spatial orientations of the segments are substantially randomly distributed with respect to one another.

In another embodiment of a method of the invention, the segments are defined in situ by vertices in the material.

In another-embodiment of a method of the invention, the segments are defined by curved portions of the material that lack vertices.

In another embodiment of a method of the invention, the step of introducing the material to conformally fill the aneurysm comprises application of a first layer of the material directly adjacent a wall of the aneurysm and a second layer substantially overlaying the first layer.

In another embodiment of the invention, a method further comprises the steps of applying additional layers until the aneurysm is substantially occluded.

In another embodiment of a method of the invention, the step of introducing the biocompatible element to fill the aneurysm comprises the deposition of the material in the manner of a viscous liquid flow.

In another embodiment of a method of the invention, the material has a stiffness preselected to produce, when the material is fully introduced into the aneurysm, a packing density in a preselected range.

In another embodiment of a method of the invention, the packing density of the biocompatible material is from at least about 10% to at least about 200%.

In another embodiment of a method of the invention, the step of introducing the biocompatible material to fill the aneurysm comprises the deposition of the material in the manner of a piece of cooked spaghetti to form a string ball in the aneurysm.

In another embodiment of the invention, a vascular occlusion device comprises a string-shaped biocompatible element having a plurality of concavities for accommodating ingrowth of vascular tissue.

In another embodiment of a device of the invention, the concavities comprise pores.

In another embodiment of a device of the invention, the concavities together form a honeycomb structure.

In another embodiment of a device of the invention, the concavities together form a reticulated porous structure.

In another embodiment of a device of the invention, the concavities comprise a plurality of fragmentary pores.

In another embodiment of the invention, a vascular occlusion device substantially excludes complete pores.

In another embodiment of a device of the invention, the concavities comprise cavities.

In another embodiment of a device of the invention, the concavities comprise concave surfaces formed in the exterior surface of a member.

In another embodiment of a device of the invention, when the member is packed into an aneurysm, concavities are positioned adjacent one another and at least some of the adjacent concavities in neighboring portions of the member together form virtual pores to accommodate tissue ingrowth.

In another embodiment of a device of the invention, wherein the average largest transverse dimension of the concavities is at least about 50 μm.

In another embodiment of a device of the invention, the average largest transverse dimension of concavities is at least about 100 μm.

In another embodiment of a device of the invention, the average largest transverse dimension of concavities is at least about 150 μm.

In another embodiment of a device of the invention, the average largest transverse dimension of concavities is at least about 200 μm.

In another embodiment of a device of the invention, the average largest transverse dimension of concavities is at least about 250 μm.

In another embodiment of a device of the invention, the average largest transverse dimension of concavities is greater than about 250 μm.

In another embodiment of a device of the invention, the average largest transverse dimension of concavities is at least about 275 μm.

In another embodiment of a device of the invention, the average largest transverse dimension of concavities is at least about 300 μm.

In another embodiment of a device of the invention, the average largest transverse dimension of concavities is greater than about 300 μm.

In another embodiment of a device of the invention, the average largest transverse dimension of concavities is not greater than about 500 μm.

In another embodiment of a device of the invention, the average largest transverse dimension of the concavities is from about 200 to about 500 microns.

In another embodiment of the invention, a vascular occlusion device comprises:

a flexible, longitudinally extending biocompatible member for delivery through a lumen of a delivery device,
the member comprising a plurality of pores having a dimensional characteristic selected on the basis of a minimum interior dimension of the lumen.

In another embodiment of a device of the invention, the interior dimension of the lumen comprises the inner diameter of the lumen, and the member has a maximum width less than the minimum interior dimension of the lumen.

In another embodiment of a device of the invention, the pore size is selected in order that the average pore diameter is greater than or equal to about 25% of the maximum width of the member.

In another embodiment of a device of the invention, the pore size is selected in order that the average pore diameter is from about 25% to about 33% of the maximum diameter of the member.

In another embodiment of the invention, a system for adjusting the properties of a longitudinally extending device comprise (a) a flexible, longitudinally extending member and (b) at least one longitudinally extending filament coupled to member (a) at various points, wherein component (b) comprises one or more materials preselected to vary at least one physical property of the device.

In another embodiment of a device of the invention, member (a) is biocompatible.

In another embodiment of a device of the invention, component (b) is selected from the group consisting of platinum, iridium, and multi-filament polymers.

In another embodiment of a device of the invention, there are at least two longitudinally extending components.

### BRIEF DESCRIPTION OF THE DRAWINGS

One or more embodiments of the invention and of making and using the invention, as well as the best mode contemplated of carrying out the invention, are described in detail below, by way of example, with reference to the accompanying drawings, in which:

FIG. 1 is a longitudinal cross-section of an artery with a saccular aneurysm;

FIG. 2 is a top view of an artery at a bifurcation;

FIG. 3 is a top view of an artery at a bifurcation with a saccular aneurysm at the point of bifurcation;

FIGS. 4 to 9 represent embodiments of implant systems useful according to the invention;

FIGS. 10 to 12 represent an embodiment of the fill/packing behavior (breaking/bending/folding) of an implant system of the invention upon deployment in an aneurysm.

FIGS. 13 to 15 represent an embodiment of a delivery system for stiffer implants according to the invention.

FIG. 16 represents an embodiment of a coaxial delivery system for softer implants;

FIGS. 17 and 18 represent an embodiment of a suture loop mechanical detachment system;

FIGS. 19 and 20 represent micrographs of tissue ingrowth;

FIGS. 21A to 21C represent different stages of embolization formation in a dog; and

FIGS. 22a to 22C are micrographs of sections of aneurysms treated with an implant of the invention.

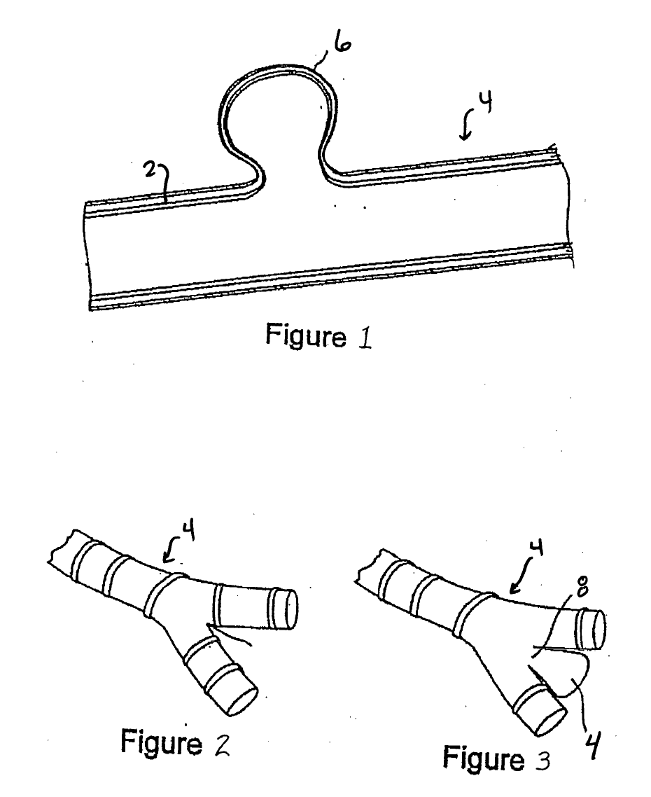

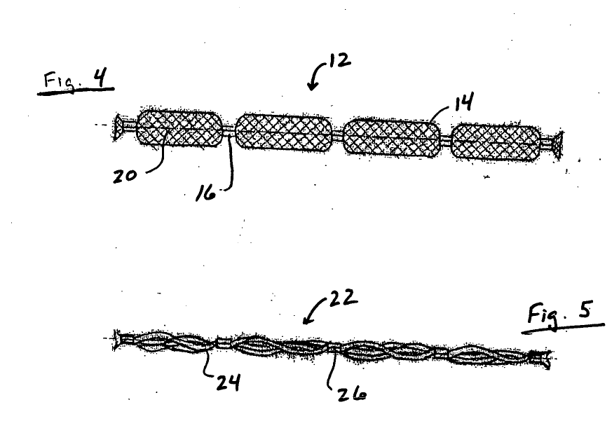

### DETAILED DESCRIPTION OF THE INVENTION

There is a need in medicine, as recognized by the present invention, for atraumatic implantable devices that can be delivered to an in vivo patient site, for example, a site in a human patient, that can occupy that site for extended periods of time without being harmful to the host. In one embodiment, such implantable devices can also eventually become biologically integrated, for example, ingrown with tissue. Various implants have long been considered potentially useful for local in situ delivery of biologically active agents and more recently have been contemplated as useful for control of endovascular conditions including potentially life-threatening conditions such as cerebral and aortic abdominal aneurysms, arterio venous malfunction, arterial embolization, or other vascular abnormalities.

The present invention relates to a system and method for treating aneurysms, particularly cerebral aneurysms, in situ and in vivo. As will be described in detail below, the present invention provides in at least one embodiment a vascular occlusion device comprising a flexible, longitudinally extending biocompatible member and one or more longitudinally extending components coupled to the biocompatible member. In another embodiment of the invention an aneurysm treatment device comprises a reticulated, biodurable elastomeric matrix implant designed to be permanently inserted into an aneurysm with the assistance of an intravascular catheter. Reticulated matrix, from which the implants are preferably made, has sufficient and required liquid permeability and thus permit blood, or other appropriate bodily fluid, and cells and tissues to access interior surfaces of the implants. This happens due to the presence of inter-connected and inter-communicating, reticulated open pores and/or voids and/or channels and/or concavities that form fluid passageways or fluid permeability providing fluid access all through. The implants described in detail below can be made in a variety of sizes and shapes, the surgeon being able to choose the best size and shape to treat a patient's aneurysm. Once inserted, the inventive aneurysm treatment device or implant is designed to cause angiographic occlusion, followed by clotting, thrombosis, and eventually bio-integration through tissue ingrowth and proliferation. Furthermore, the inventive aneurysm treatment device can carry one or more of a wide range of beneficial drugs and chemical moieties that can be released at the affected site for various treatments, such as to aid in healing, foster scarring of the aneurysm, prevent further damage, or reduce risk of treatment failure. With release of these drugs and chemicals locally, employing the devices and methods of the invention, their systemic side effects are reduced.

An implant or occlusion device according to at least one embodiment of the invention comprises a reticulated biodurable elastomeric matrix or other suitable material and structural filaments and can be designed to be inserted into an aneurysm through a catheter. A preferred reticulated elastomeric matrix is an optionally compressible, lightweight material, designed for its ability to expand preferably in conformal fashion within the aneurysm without expanding too much and tearing the aneurysm. In another embodiment, preferred reticulated elastomeric matrix is an optionally compressible, lightweight material, designed for its ability to pack preferably in conformal fashion within the aneurysm without expanding or without any significant expansion and without tearing the aneurysm. Although multiple implants can be deployed, used, or implanted, preferably five or less implants should fill the aneurysm to achieve angiographic occlusion. In another embodiment, preferably ten or less implants should fill the aneurysm to achieve angiographic occlusion. The ratio of implant (or implants) volume to aneurysm volume is defined as packing density. It is contemplated, in one embodiment, that even when their pores become partially filled or completely filled with biological fluids, bodily fluids and/or tissue in the course of time or immediately after delivery, and/or the implants are either still partially compressed or partially recovered after delivery, such implantable device or devices for vascular malformation applications have a volume of at least about 10% of the aneurysm volume. In another embodiment, such implantable device or devices for vascular malformation applications have a volume, prior to packing in vivo, of at least about 25% of the aneurysm volume. In another embodiment, such implantable device or devices for vascular malformation applications have a volume, prior to packing in vivo, of at least about 50% of the aneurysm volume. In another embodiment, such implantable device or devices for vascular malformation applications have a volume, prior to packing in vivo, of at least about 75% of the aneurysm volume. In another embodiment, such implantable device or devices for vascular malformation applications have a volume, prior to packing in vivo, of at least about 100% of the aneurysm volume. In another embodiment, such implantable device or devices for vascular malformation applications have a volume, prior to packing in vivo, of at least about 125% of the aneurysm volume. In another embodiment, such implantable device or devices for vascular malformation applications have a volume, prior to packing in vivo, of at least about 175% of the aneurysm volume. In another embodiment, such implantable device or devices for vascular malformation applications have a volume, prior to packing in vivo, of at least about 200% of the aneurysm volume. Insertion of the implant followed by tissue ingrowth should result in total obliteration of the aneurysm sac.

It would be desirable to have an implantable system which, e.g., can optionally cause immediate thrombotic response leading to clot formation, and eventually lead to fibrosis, i.e., allow for and stimulate natural cellular ingrowth and proliferation into vascular malformations and the void space of implantable devices located in vascular malformations, such as a cerebral aneurysm, and to stabilize and possibly seal off such vascular abnormalities in a biologically sound, effective and lasting manner.

In another embodiment of the invention, cellular entities such as fibroblasts and tissues can invade and grow into a reticulated elastomeric matrix. In due course, such ingrowth can extend into the interior pores and interstices of the inserted reticulated elastomeric matrix. Eventually, the elastomeric matrix can become substantially filled with proliferating cellular ingrowth that provides a mass that can occupy the site or the void spaces in it. The types of tissue ingrowth possible include, but are not limited to, fibrous tissues and endothelial tissues.

In another embodiment of the invention, the implantable device or device system causes cellular ingrowth and proliferation throughout the site, throughout the site boundary, or through some of the exposed surfaces, thereby sealing the site. Over time, this induced fibrovascular entity resulting from tissue ingrowth can cause the implantable device to be incorporated into the aneurysm wall. Tissue ingrowth can lead to very effective resistance to migration of the implantable device over time. It may also prevent recanalization of the aneurysm. In another embodiment, the tissue ingrowth is scar tissue which can be long-lasting, innocuous and/or mechanically stable. In another embodiment, over the course of time, for example, for from about 2 weeks to about 3 months to about 1 year, implanted reticulated elastomeric matrix becomes completely filled and/or encapsulated by tissue, fibrous tissue, scar tissue or the like.

The invention has been described herein with regard to its applicability to aneurysms, particularly cerebral aneurysms. It should be appreciated that the features of the implantable device, its functionality, and interaction with an aneurysm cavity, as indicated above, can be useful in treating a number of arteriovenous malformations (“AVM”) or other vascular abnormalities. These include AVMs, anomalies of feeding and draining veins, arteriovenous fistulas, e.g., anomalies of large arteriovenous connections, and abdominal aortic aneurysm endograft endoleaks (e.g., inferior mesenteric arteries and lumbar arteries associated with the development of Type II endoleaks in endograft patients). Other embodiments include reticulated, biodurable elastomeric implants for in vivo delivery via catheter, endoscope, arthroscope, laparoscope, cystoscope, syringe or other suitable delivery-device and can be satisfactorily implanted or otherwise exposed to living tissue and fluids for extended periods of time, for example, at least 29 days.

Shaping and sizing can include custom shaping and sizing to match an implantable device to a specific treatment site in a specific patient, as determined by imaging or other techniques known to those in the art. In particular, one or at least two comprise an implantable device system for treating an undesired cavity, for example, a vascular malformation.

Employment of an implant that can support invasion of fibroblasts and other cells enables the implant to eventually become a biointegrated part of the healed aneurysm. Elastin, fibrin, or other suitable clot-inducing material can also be coated onto the implant providing an additional route of clot formation.

In one embodiment of the invention the implant can also contain one or more radiopaque markers for visualization by radiography or ultrasound to determine the orientation and location of the implant within the aneurysm sac. Preferably plantinum markers are incorporated in the implant and/or relevant positions of delivery members.

If desired, the outer surfaces of the implant or occlusion device can be coated, after fabrication of the implant or occlusion device with functional agents, such as those described herein, optionally employing an adjuvant that secures the functional agents to the surfaces and to reticulated elastomeric matrix pores adjacent the outer surfaces, where the agents will become quickly available. The functional agents can be coated, during the fabrication of the implant or occlusion device. Such external coatings, which may be distinguished from internal coatings provided within and preferably throughout the pores of reticulated elastomeric matrix used, may comprise fibrin and/or other agents to promote fibroblast growth.

Once an aneurysm has been identified using suitable imaging technology, such as a magnetic resonance image (MRI), computerized tomography scan (CT Scan), x-ray imaging with contrast material or ultrasound, the surgeon chooses which implant he or she feels would best suit the aneurysm, both in shape and size. The chosen implant is then loaded into an intravascular catheter in a linear or uncompressed state. The implants can be sold in a sterile package containing a pre-compressed or slightly compressed or uncompressed implant that is loaded into a delivery catheter. Alternatively, the implant can be sold in a sterile package in a linear or uncompressed state, and the surgeon at the site of implantation can use a suitable loading device, e.g. a ring, funnel or chute for loading into the catheter with or without application of compression.

After an implant according to the invention is loaded into the catheter, the catheter is then advanced through an artery to the diseased portion of the affected artery using any of the techniques known in the art. With use of the catheter the implant of the invention is then inserted and positioned within the aneurysm, the implant filling the aneurysm by bending and folding on itself within the sac. The implant, in an embodiment of the invention, preferably fills the sac conformally, due to inherent properties of the device comprising elastomeric matrix and structural filaments, which properties include properties that allow the device to fold and pack as it fills an aneurysm sac. In another embodiment, the implant according to the invention preferably fills the sac conformally, due to inherent properties of the device comprising viscoelastic and preferably matrix and structural filaments, with properties that allow the device to fold and pack as it fills an aneurysm sac. Properties of the device, in various embodiments, permit the formation of one or more vertices that permit the device to adopt geometries that are non-curvilinear or that otherwise include one or more points at which the device can “break”, fold or otherwise form angles, bends or discretizations, or very small radii of curvature. Properties that permit these sorts of formations, and others according to the present invention, may be conferred by any of a variety of features, including topological features, including but not limited to crimps, the imposition or interaction of additional members or materials, such as filaments, sutures, staples, adhesives, or other additional features or materials without limitation. Embodiments of the device can pack while folding onto itself like in cooked spaghetti, a metallic chain, a thread of honey, or other material capable, for example, of adopting sharp direction changes prior to, during, or after introduction into an aneurysm, malformation or other target structure. The device can also pack following a random or irregular curvature or in another case following a more regular curvatures that, for example, resemble helical configurations. The assembly can be enhanced by periodic notches along the length of the assembly forming natural “breaking points,” as well as the platinum (or other) marker band crimping, or other structures as described herein, which further enhances the breaking behavior or other formation of vertices that permit advantageous packing geometries. A non-compressed or slightly compressed linear implant according to the invention will be advanced to conformally fill the aneurysm sac.

When properly located in situ, pursuant to the teachings of this invention, implants or occlusion devices are intended to cause angiographic occlusion of the aneurysm sac. The presence of implants or occlusion devices, optionally including one or more pharmacologic agents borne on each implant, stimulates fibroblast proliferation, growth of scar tissue around the implants, and eventual immobilization of the aneurysm.

Advantageously, the implants of the invention can, if desired, comprise reticulated biodurable elastomeric implants having a materials chemistry and microstructure as described herein.

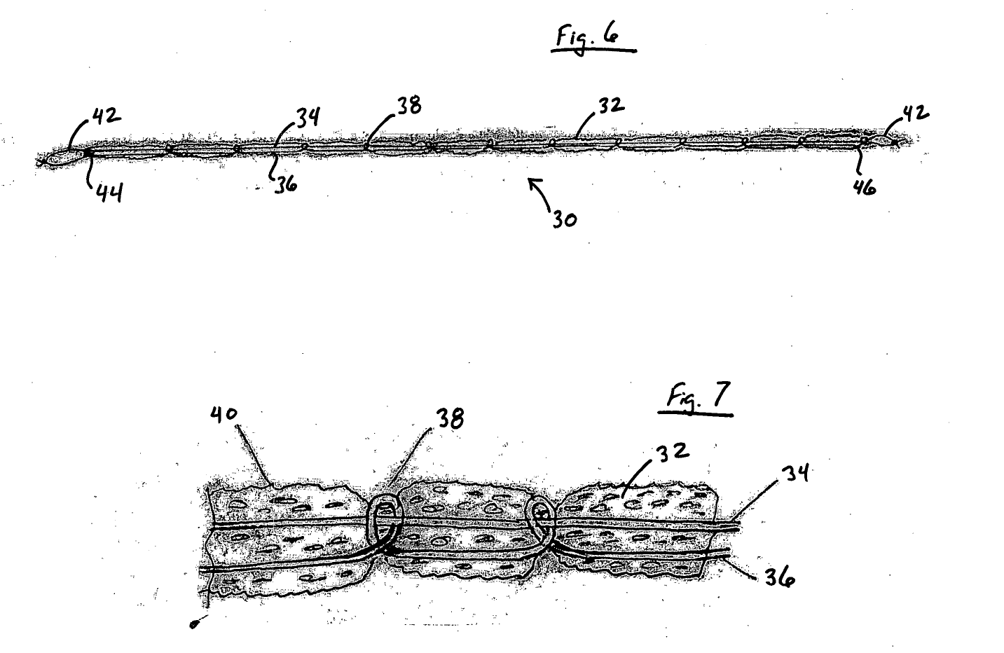

The invention can perhaps be better appreciated from the drawings. In FIG. 4, an implant 12 is formed preferably from a biodurable reticulated elastomeric matrix 14 optionally having a regular cross-section such as round, square, ellipsoidal, triangular, rectangular or other multi-sided polygonal cross-sections. In another embodiment, the cross-section of biodurable reticulated elastomeric matrix 14 can be of an irregular shape or random. In yet another embodiment, the cross-section of biodurable reticulated elastomeric matrix 14 can be of regular cross-section for part of the length of implant 12 and can be of irregular cross-section for part of the length of implant 12, that is, a combination of regular cross-section and irregular cross-section. In yet another embodiment, the topology of biodurable reticulated elastomeric matrix 14 can be of regular cross-section for part of the length of implant 12 and can be of irregular cross-section for part of the length of implant 12, that is, a combination of regular cross-section and irregular cross-section. Radiopaque, preferably platinum, markers 16 are positioned or crimped every about 2 to about 10 mm to form a chain link, noodle-like, or other structure consistent with the principles of this and other aspects of the invention.

In certain embodiments, implant 12 has a structural filament 20 extending through the entire length of implant 12 to prevent implant 12 from jamming, tearing, balling, breaking, or fragmenting, to provide support for pulling and/or pushing during delivery or deployment, and to prevent migration during delivery or deployment. The structural filament, which is attached to the matrix or incorporated into the matrix, comprises preferably a single material such as metal or polymer or in other cases a combination of both. Without being bound by any particular theory, the structural filament provides scaffold or support structure to the implant of the invention, without which the device will tend to buckle during delivery or fold onto itself during storage and handling. This is due to the small cross-sectional area and large length to diameter ratio (or length to any characteristic dimension defining the cross-section) of the implant of the invention whether it is made from reticulated elastomeric matrix or any other viscoelastic thermoplastic or viscoelastic thermoset cross-linked polymeric material. Additionally, the flexibility of the matrix material in most cases without the presence of the structural member will make the device buckle during delivery or fold onto itself during storage, handling, and delivery. The other thermoplastic or thermoset cross-linked polymeric material can be either synthetic or naturally occurring. The need for the structural filament is especially true in the case of materials containing a large amount of voids such as reticulated elastomeric matrix as the inherent mechanical properties of the these structures are low due to the presence of high void content and to their inter-connected and inter-communicating open pore structures, features that support tissue ingrowth and proliferation and eventual bio-integration of the implant to the aneurysm site. Thus the device or devices of this invention while offering sufficient column strength or rigidity or biomechanical integrity for advancement through a catheter or microcatheter, at the same time cannot be too stiff or too rigid so that they are unable to still fold and pack in order to provide a superior packing or filling of the aneurysm on delivery to the aneurysm site.

Structural filament 20 can be biosorbable or non-resorbable and can comprise a polymer such as polyester, a metal such as platinum, or a combination thereof, including, but not limited to, known suture materials or suture composites. In another embodiment of the invention, the metal can be radiopaque. Moreover, structural filament 20 can be a monofilament fiber, co-mingled fibers, knotted, twisted, braided rope, wire, cable composite scaffold, mesh, woven mesh or knitted mesh. In another embodiment of the invention, filament 20 can be a braided subassembly. In a another embodiment of the invention filament 20 is polymeric fiber, carbon fiber, glass fiber, synthetic polymeric fiber or filament, a single platinum wire, other metallic fiber, a twist or braid of platinum wire and polymeric fiber or filament, or twisted or braided double platinum wires or combinations thereof. In another embodiment filament 20 can be a monofilament. In another embodiment, filament 20 can be a multifilament In another embodiment, filament 20 can be a reinforcing element. The length of implant 12 could be from about 5 mm to about 800 mm, preferably from about 50 mm to about 600 mm, and the diameter or effective diameter or any dimension or dimensions characteristic of the cross-section could be from about 0.25 mm to about 10 mm, preferably from about 0.50 mm to about 2 mm. In cases where the structural filament 20 can be biosorbable, as the structural filament degrades over time, it may make more of the cross-section accessible to tissue ingrowth and proliferation.

A matrix according to the present invention, such as the polymeric matrix, which is biodurable, elastomeric, and reticulated, together with the one or more structural filaments embedded in or incorporated into the matrix, forms an embodiment of the implant of the invention. This structure has a number of advantages when it is used to fill an irregularly shaped aneurysm sac. In certain embodiments, the presence of the one or more structural filaments, when imbedded or incorporated in the matrix, enhances the propensity of the implant to form coil-like shapes that allows it to pack in an easier fashion and fill the aneurysm and in the process allows the implant to conformally fill the sac in a way that conforms in a superior fashion to the internal shape and volume of the sac. In other embodiments of the invention, the presence of the one or more structural filaments, when imbedded or incorporated into the matrix, enhances the propensity of the implant to fold onto itself like spaghetti, chain, a thread of honey or the like, and allows it to pack and fill the aneurysm, in the process allowing the implant to conformally fill the sac in a way that conforms in a superior fashion to the internal shape and volume of the sac. In another embodiment, the device can pack while folding onto itself like such that the deposition of the material in the manner of a piece of cooked spaghetti to form a string ball in the aneurysm. The device can pack following random or irregular curvatures or in another case following more regular curvatures that, for example, resemble helical configurations. In embodiments of the invention, as also described above, the presence of the one or more material, topological or other features or structures, such as structural filaments, when imposed, imbedded or incorporated in the matrix, create or enhance a propensity of the implant to form vertices, such as folds, angles, discretized, or non-curvilinear geometries, or very low radii of curvature, and to also form shapes containing curvatures allowing the implant to conformally fill the sac in a way that conforms in a superior fashion to the internal shape and volume of the sac. In certain embodiments, implants or devices according to this aspect of the present invention can be applied in actual or virtual layers, being deposited in a manner akin to strokes of a paintbrush or other suitable insertion or deposition techniques. Implants or devices that have a propensity to form shapes containing curvatures and/or those that fold onto itself optionally can be compressed as they make contact during delivery with other parts of itself or with other delivered devices in the aneurysm or with wall of the aneurysm thereby making it easier to pack in a superior fashion. This progressive compression of the device allows for superior packing, as the device is able to fill small regions of the previously unfilled aneurysm sac. This is better appreciated when the use and availability of different soft and ultra soft devices, that facilitates and enhances this superior packing towards the end of the procedure of implanation, and will be presented and discussed below. The device, or portions of it, may in certain embodiments, including but not limited to generally stringlike or other elongate forms, be formed in order to otherwise lack a predetermined shape or geometry in order to enhance its conformal filling behavior and resultant superior packing density.

The presence of the one or more structural filaments also prevents jamming, tearing, balling, breaking, or fragmenting, to provide support for pulling and/or pushing during delivery or deployment, and to prevent migration during delivery or deployment. Without being bound by any particular theory, the absolute or comparative stiffness of the structural members in relation to the matrix in certain embodiments allows these additional advantages. It is believed that additional periodic material or topological features, including but not limited to crimps or notches along the length of the implant, or shapes, couplings, or other relationships between components of the device that can be formed along its length, as described above, permit the member to be modulated, inserted, and/or deposited in a conformal or other desired geometry with respect to the target structure. In one embodiment, such feature(s) may the optionally add other features, such as making the device radiopaque in certain embodiments by crimping platinum or other marker bands along the length of the device to form a structure that also preferably forms vertices (as described above) or otherwise folds at or around these periodic notches and/or crimped platinum marker bands allowing the implant to fill the sac in a way that conforms in a superior fashion to the internal shape and volume of the sac. The overall resultant phenomenon is again similar to that of cooked spaghetti filling a bowl, for example, and the folded spaghetti-like structure of an embodiment of the implant of the invention provides more complete packing of the aneurysm sac when compared to platinum coils or other polymeric devices that are pre-formed or imparted with a shape prior to delivery to the aneurysm sac. The resulting packing is more complete or tight and is much less likely to have voids or unfilled space when compared to platinum coils or other polymeric devices that are pre-formed or imparted with a shape prior to delivery to the aneurysm sac. In certain embodiments of this inventionthe column strength or rigidity or biomechanical integrity device or devices of this invention can be engineered and controlled to facilitate delivery for their advancement through a tortuous catheter or microcatheter and at the same time not make the devices too stiff or too rigid so that they are unable to still fold and pack in order to provide a superior packing or filling of the aneurysm on delivery to the aneurysm site.

The implant 22 in FIG. 5 comprises two or more, preferably from about 3 to 6, cylindrical or string segments 24 that are held together by a structural filament (not shown) or marker 26 for structural integrity for delivery or deployment or to be blended with other components. As with implant 12, radiopaque markers 26 are crimped from about 2 to about 10 mm apart. The length and effective diameter of implant 22 are approximately the same as those of implant 12.

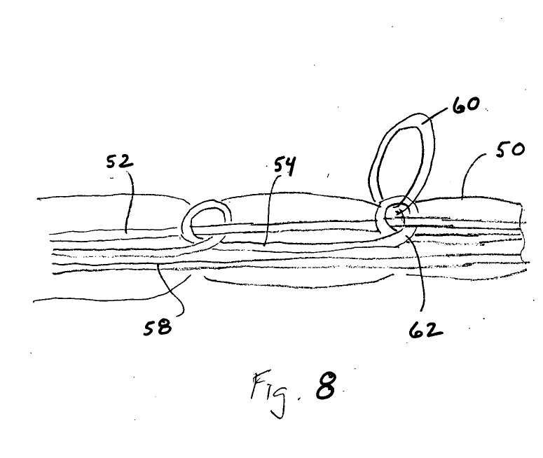

A preferred embodiment of an implant 30, also known as a NEUROSTRING™ implant,” is shown in FIGS. 6 to 9. Implant 30 is formed from an elastomeric matrix member 32 having a round, square, ellipsoidal, multi-sided, polygonal, or rectangular, but preferably round, cross-section. In another embodiment, implant 30 is formed from an elastomeric matrix member 32 having an irregular shape. In yet another embodiment, the cross-section of elastomeric matrix 32 can be of regular cross-section for part of the length of implant 30 and can be of irregular cross-section for part of the length of implant 30, that is, a combination of regular cross-section and irregular cross-section. In yet another embodiment, the topology of biodurable reticulated elastomeric matrix 32 can be of regular cross-section for part of the length of implant 30 and can be of irregular cross-section for part of the length of implant 30, that is, a combination of regular cross-section and irregular cross-section. In another embodiment, matrix member 32 is biodurable and reticulated. Two longitudinally extending, essentially parallel structural filaments 34 and 36 extend the length of implant 30, and at regular intervals structural filaments 34 and 36 form knots or ropes 38 that define matrix subsections 40. A purpose of the knots is to secure the structural filament to the elastomeric matrix. This can be seen more clearly in the detail of FIG. 7. Other means of incorporating filaments 34 and 36 into matrix 32 that causes similar attachment are commonly known, for example, sewing stitches. The respective ends of structural filaments 34 and 36 form a loop 42 at the proximal end 44 and optionally also distal end 46, of implant 30.

Structural filaments 34 and 36 can be biosorbable or non-resorbable, preferably non-resorbable, and comprised of a polymer such as polyester, a radiopaque metal such as platinum, or a combination thereof, including, but not limited to, known polymeric fiber or filament materials or polymeric fiber or filament composites. Moreover, reinforcing filaments 34 and 36 can each be a monofilament, braided rope or wire, or a wire or cable.

In an embodiment of the invention, one or more elongate structural members in the device, such as filaments, may be included and, if so. May be provided with features or coupled to one another to confer desired properties. Filament 34 may be polymeric fiber, carbon fiber, glass fiber, synthetic suture, a single platinum wire, other metallic fiber, a twist or braid of platinum wire and polymeric fiber or filament, or twisted or braided double platinum wires or other materials or combinations thereof. Filament 36 is polymeric fiber or other filament such as are described above. Filament 34 or filament 20 can also be a monofilament fiber, co-mingled fibers, knotted, twisted braided rope, wire, a cable, composite scaffold, mesh, woven mesh or knitted mesh, or other material, structure or combination. In one embodiment according to the invention, filament 34 can be a structural element. Filament 34 may comprise a subassembly prepared using a coil winder and separate spools of fibers used to make polymeric fibers or filaments and platinum wires of differing thicknesses, thereby creating a twisted rope-like composite subassembly with varying stiffness and radiopacity. Known methods such as braiding may also be used to create such a subassembly. In another embodiment of the invention, the components of filament 34 may be available on separate spools or spindles and the final structural element can be formed during the attachment or the incorporation of the matrix member 32 to filament 34. In another embodiment, filament 34 may comprise a sub-assembly in which a platinum micro coil string wound from platinum micro wire, over a fiber core or over the platinum wire core, will provide more integrity for pull/push action including good radiopacity. Construction or fabrication of filament 34 can be achieved in, for example, by using a sewing machine. Instead of using twisted platinum wire with fiber into braid and than loaded into sawing machine, in one embodiment a regular coil having inner core fiber or platinum wire is then loaded into the sewing machine to get knotted with the second sewing machine polymeric fiber or filament or wire string.

The platinum wire useful according to the invention preferably has a diameter of from about 0.0005 in. to about 0.005 in., more preferably from about 0.001 in. to about 0.003 in. Suitable platinum wire is available from sources such as Sigmund Cohn Corp. The fibers useful according to the invention comprise commercially available, non-absorbable polymeric fibers used to make suture fiber or filament having an effective diameter of from about 0.0005 in. to about 0.010 in., preferably from about 0.010 in. to about 0.005 in. Preferably the fibers are available on spools and have compositions and diameters comparable to commercially available sutures, for example, sutures available from Johnson & Johnson under the name ETHIBOND EXCEL®, PROLENE®, ETHILON®, Coated VICRYL®, or MONOCRYL®.

Varying the structural filaments results in implants according to the invention having different characteristics. When each of the filaments is polymeric fiber or filament, the resulting implant is “Ultra Soft”, as set forth in the table below. When at least one of the filaments includes platinum wire, the resulting implant is “Soft” or “Stiff”. The stiffness of the device can be measured by the slope of the load versus extension curves during an uniaxial tensile pull using a tensile testing machine and can be in the range of from 1 to 200 pounds per inch (0.18 N/mm to 35 N/mm), preferably in the range of from 5 to 100 pounds per inch (0.88 N/mm to 18 N/mm). The breaking strength of the device can be measured during an uniaxial tensile pull using a tensile testing machine and can be in the range of from approximately 0.05 to 23 pounds (0.2 to 100 Newtons) and preferably in the range of approximately 0.05 to 7 pounds (1.0 to 30 Newtons).

TABLE 1

| Filament 204 | Filament 206 | Resulting Implant stiffness & |
|---|---|---|
| (bottom bobbin) | (top bobbin) | functionality |
| Fiber equivalent to (7-0) | Fiber equivalent to | “Ultra Soft” implant for finishing (filling |
| Suture with approx. | (7-0) suture with | residual gaps in aneurysm), requires core |
| diameter = 0.05 mm | approx. diameter = 0.05 mm | wire/coaxial pusher or hydraulic assistance |
| | | for delivery. |
| Single Pt wire | Fiber equivalent to | “Soft” implant for framing and filling the |
| (0.002-0.005”) | (7-0) suture with | aneurysm, can be pushed from proximal |
| | approx. diameter = 0.05 mm | end using a pusher member. |
| Twisted or braided | Fiber equivalent to | |
| Pt wire + Fiber equivalent | (7-0) suture with | |
| to (7-0) Suture with | approx. diameter = 0.05 mm | |
| approx. diameter = 0.05 mm | | |
| Twisted or braided | Fiber equivalent to | “Stiff” implant for framing the aneurysm, |
| Double Pt wire | (7-0) suture with | can be pushed from proximal end using a |
| | approx. diameter = 0.05 mm | pusher member. |

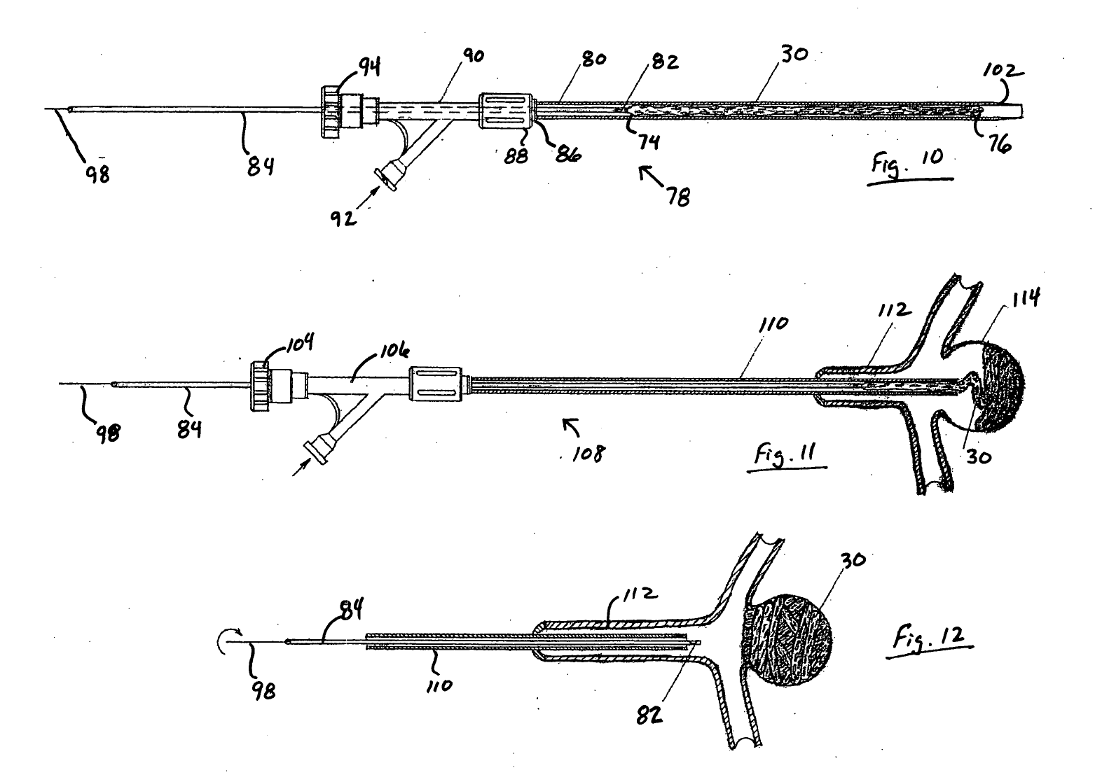

It is preferred to include additional platinum markers to be crimped in from 1 to 10 mm sequences to provide safe radiopacity/visibility. Framing coils or a stent may be used to prevent migration. Delivery of the Ultra Soft implant requires use of the supporting core-wire delivery system described below in FIG. 16 or hydraulic injection with a syringe. Delivery of the Soft or Stiff implants requires a pusher member as described below in FIGS. 10, 11, and 12.

When according to the invention one structural filament is a platinum wire and the other structural filament is polymeric fiber or filament, the resulting implant behaves like a coil to form helical packing during deployment into the aneurysm sac. A significant difference between an implant of the invention and a coil is that the implant of the invention does not have a predetermined memory, as does a coil. Also, the implant of the invention is malleable and will conform to the dimensions of the aneurysm sac. The stiffness can be controlled by varying the diameter of the platinum wire or the structure, as shown above, and the filament structure can act as a framing structure in lieu of the framing coils or stent necessary with a softer implant. The stiffness can also be controlled by varying the number of platinum wires used. The stiffer implants function to prevent migration and to facilitate better packing of the aneurysm sac, while the softer versions can be used as filler material to optimally embolize the aneurysm. This stiffer implant may be more useful for different vessel occlusion applications within the body. Delivery of the stiffer implant can be accomplished with a regular delivery system not having a supporting core-wire mandrel or hydraulic injection.

It is within the scope of the invention that each filament can be a platinum wire. The resulting implant will be similar to the implant described above but slightly stiffer and more radiopaque.

In an embodiment of the invention implant 30 has regularly spaced radiopaque markers that are attached to every second to every sixth knot, preferably every third or fourth knot. These radiopaque markers tend to encourage the chain-like behavior that is characteristic of this embodiment. Notching of the elastomeric matrix/structural assembly and optional periodic crimping of platinum marker bands will allow the implant of the invention to bend and fold when deployed in an aneurysm and break like a chain. This bending and folding allows the implant to conformally fill the aneurysm sac like a liquid when deployed from the microcatheter. The overall resultant phenomenon is again similar to that of spaghetti filling a bowl or a metallic chain folding onto itself. In certain cases the implant fills the aneurysm sac in a manner similar to that of very viscous liquid flow. When multiple implants are placed in an aneurysm, the implants or devices form shapes containing curvatures or those that fold onto themselves optionally can be compressed further as they make contact during delivery with themselves or with other delivered devices in the aneurysm or with wall of the aneurysm, thereby making it easier to pack in a superior fashion. Platinum marker bands will impart additional radiopacity.

In another embodiment of the invention radiopaque microstaples instead of the radiopaque markers could be regularly spaced along the length of the implant every second to every sixth knot. This configuration would also encourage chain-like behavior.

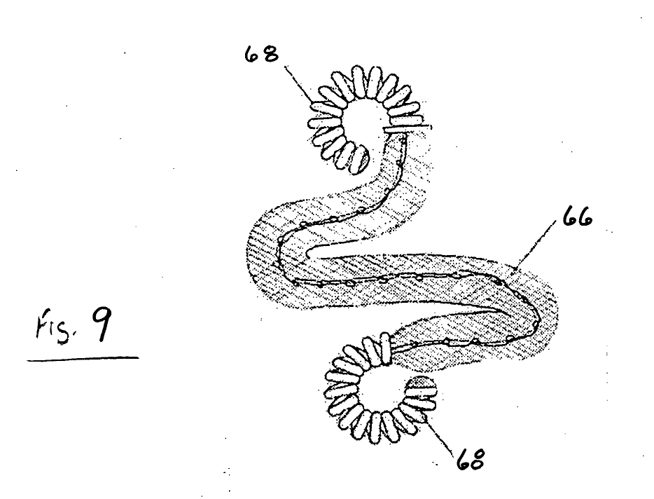

In another embodiment of the invention which is shown in detail in FIG. 8, an implant 50 has filaments 52 and 54 similar to the structure described above but with an additional filament 58. Filament 58 comprises platinum wire or polymeric fiber or filament and provides additional structural integrity. Optionally implant 50 may have loops 60 attached to every fourth to twentieth knot 62. Loops 60 comprise polymeric fiber or filament or physiologically acceptable, optionally radiopaque, metal such as platinum wire. Loops 60 are used to attach Ultra Soft implants according to the invention to the core wire/coaxial pusher during delivery.

In the embodiment of the invention shown in FIG. 9, an implant 66 has proximal and/or distal, preferably radiopaque, coils 68. Coils 68 preferably are non-linear in an unstressed state. For example, when an implant having such coils is advanced into an aneurysm, the coils, especially the distal coil, will assist is conformally filling the aneurysm. The distal coil, that is, the first coil out of the microcatheter, functions to start the implant breaking in the aneurysm sac. When this coil hits the wall, it curls on itself into a half-loop, which initiates the breaking behavior of the implant which follows. The proximal coil, that is, the last coil out of the microcatheter before detachment, serves as a visual “end point” to the operator that he/she has deployed the end of the implant. This is advantageous in providing a clear “start” & “stop” visual marker system which other implants don't have.

The length of implant 30 or 50 could be from about 5 mm to about 1500 mm, preferably from about 1 cm to about 50 cm, and the diameter or effective diameter could be from about 0.25 mm to about 12 mm, preferably from about 0.250 mm to about 0.5 mm. The defined sections of the implant are each from about 0.5 mm to about 1 cm in length. The implant of the invention is delivered in an uncompressed state. Also, the reticulated elastomeric matrix and the structural filaments are intertwined or the latter is incorporated into the former so that they, in effect, work together, more notably that the structural filaments provide support to the elastomeric matrix. There should be at least from about 1 to about 4 pores of reticulated elastomeric matrix material surrounding the structural filaments, even after trimming or shaving the elastomeric matrix material as described below. In another embodiment, there should be at least from about 1 to about 10 pores of reticulated elastomeric matrix material surrounding the structural filaments, even after trimming or shaving the elastomeric matrix material as described below.

During the shaving or trimming some of the pores may open to form concavities, that is, any structure having at least one concave surface feature, that may or may not be fully contained within the implant or may intersect an outer surface of the implant, which may have a dimension greater than the maximum diameter of the implant, and which may encompass pores, partial or fragmentary pores, cavities that alone, or combined to form “virtual” pores, accommodate tissue ingrowth. Such structure also encompasses honeycomb structures, which may comprise a plurality of fully and/or partially contained concavities in the form of pores, and a skeleton or framework of a reticulated foam, in which the concave partial surfaces remain or are formed after an implant is shaved or trimmed to its final or operative width.

The number of pores present after shaving or trimming may inversely correlate to the pore size of the material in that there will be a greater number of pores remaining in material with a smaller pore size. When deployed in the aneurysm, the implant of the invention bends and folds (plicates), creating a conformal “foam ball” that serves as a porous scaffold for tissue ingrowth. Even though each individual string may only have 4 to 5 pores, optionally from 2 to 10 pores, the plication of the implant allows creation of a “solid” conformal scaffold.

According to the invention the structural filaments can be inserted into an elastomeric matrix by hand or by mechanical means such as a mechanical stitching or sewing machine. Preferably a commercial sewing machine having two bobbins is used where each bobbin has filament material. In a preferred embodiment one bobbin has a braided platinum wire/polymeric fiber or filament filament subassembly and a second bobbin has polymeric fiber or filament. These subassemblies are then sewn into elastomeric matrix sheet material of from about 1 to about 2 mm thickness to create a continuous stitch line from about 10 to about 50 cm long. In another embodiment of the invention, an adhesive can be used to adhere a single structural filament to the elastomeric matrix, such as by dipping or polymerizing the adhesive to the structural filament. The elastomeric matrix is then carefully trimmed or shaved by hand to a desired diameter. The outer diameter of each elastomeric matrix section should be equal to or slightly less than the inner diameter of the corresponding introducer sheath, discussed below.

A system 78 for the delivery of a Soft or Stiff implant according to the invention such as implant 30 or other implants according to the invention is shown in FIG. 10. Proximal end 74 of implant 30 is engaged within an introducer sheath 80 by the distal end 82 of a pusher rod or member 84. The proximal end 86 of sheath 80 engages the distal portion 88 of a manifold or side arm 90, which has an opening 92 for continuous flush. Pusher member 84 extends proximally through valve 94, and pusher member 84 has a lumen (not shown) which receives an interlocking wire 98, which provides support to pusher member 84 and helps retain implant 30.

For delivery of implant 30 or another occlusion device according to the invention to a patient, a flushing solution such as saline solution is introduced into opening 92 of system 78 to remove air and straighten out implant 30. Then, the tapered distal tip 102 of sheath 80 is introduced with continuous flushing into the hemostastis valve 104 of a side arm 106 of a microcatheter assembly 108 such as is shown in FIG. 11. Sheath 80 is inserted into microcatheter 110, after which sheath 80 and side arm 90 are withdrawn, leaving implant 30, pusher member 84, and interlocking wire 98.

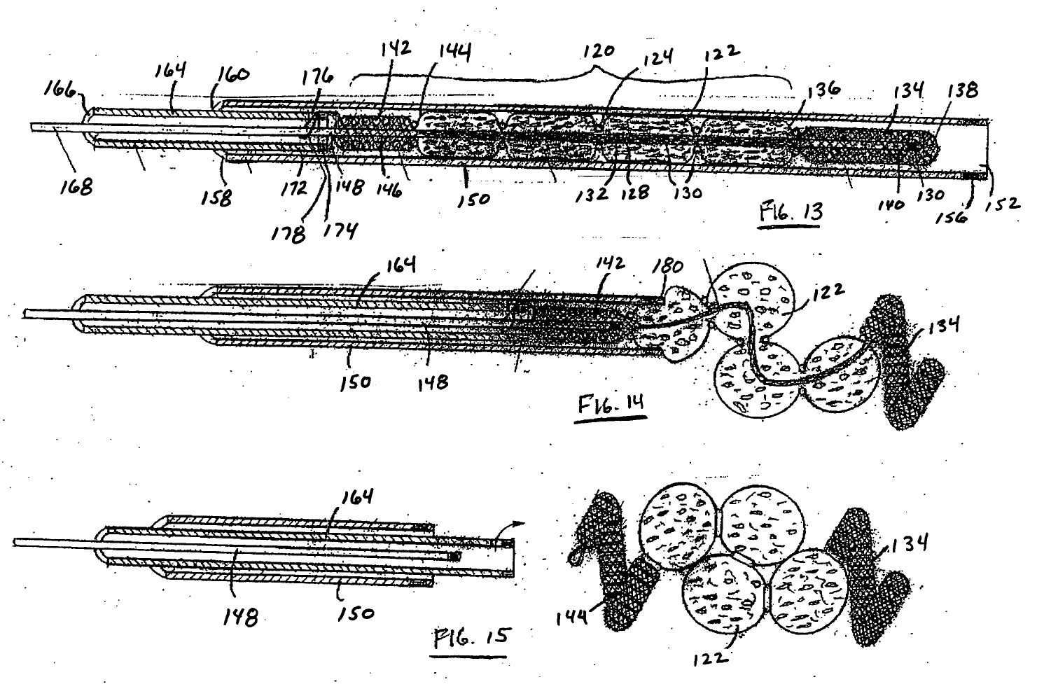

Delivery of implant 30 is shown in FIGS. 11 and 12, where the distal end 76 of implant 30 is advanced through microcatheter 110 and through an artery 112 to a position adjacent an aneurysm 114. Implant 30 is advanced further to fill aneurysm 114. When aneurysm 114 has been filled, as shown in FIG. 12, the distal end 82 of pusher rod 84 is disengaged from implant 30 and withdrawn through microcatheter 110.

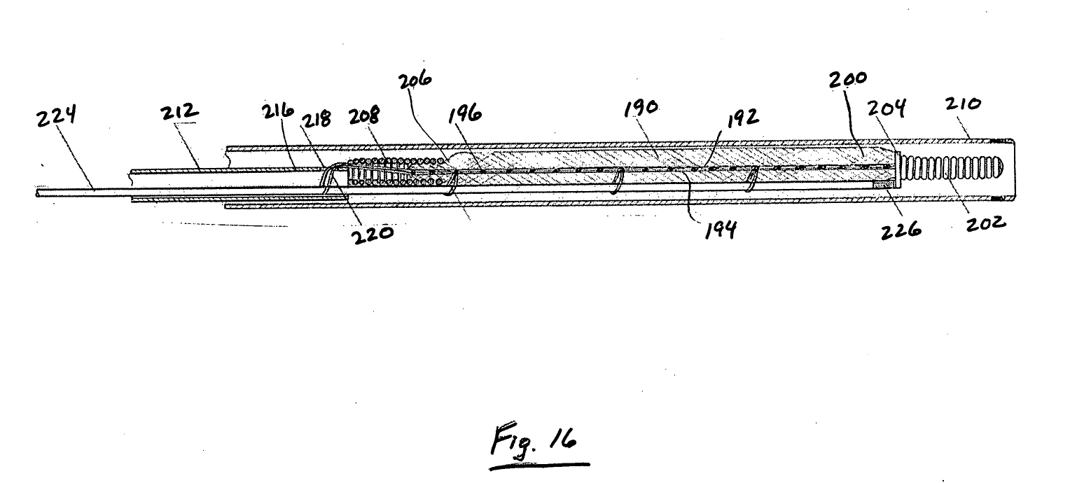

In another embodiment of the invention shown in FIGS. 13 to 14, the delivery of an expandable implant according to the invention is shown. An elastomeric structure 120 comprises two or more sections 122, preferably from about 2 to about 100, that are defined by radiopaque rings, e.g., platinum rings or compression members 124 or similar mechanisms. Elastomeric sections 122 comprise a longitudinally extending flexible mesh 128 defining a lumen 132. A distal spring section 134 attached to the distal end 136 of structure 120 comprises a distal tip 138 and a lumen 140 in communication with lumen 132. At the proximal end 144 of structure 120 a proximal spring 142 is attached to proximal end 144 and has a lumen 146 extending therethrough. A flexible but rigid wire 148 extends through lumen 146, lumen 132, and lumen 140. Wire 148 has a radiopaque tip marker 130. Flexible mesh 128 extends distally as a jacket to cover coil 134 and proximally as a jacket to cover coil 142.

Compressed structure 120 is positioned within a delivery catheter 150 that has a longitudinally extending lumen 152 and a distal radiopaque marker 156. The proximal end 158 of catheter 150 has a narrowed opening 160 that slidably engages a pushing catheter 164.

The proximal end 166 of pushing catheter 164 slidably engages the proximal section 168 of wire 148. The distal end 172 of pushing catheter 164 comprises a radiopaque marker 174 and an opening 176. A flexible loop or wire 178 attached to coil 146 extends through opening 176 to engage wire 148.

To deploy structure 120, as shown in FIG. 14, pusher catheter 164 and wire 148 are advanced distally. As portions of structure 120 extend distally past the distal end 180 of delivery catheter 150, wire 148 is withdrawn in the proximal direction. Eventually, as shown in FIG. 8, wire 148 is withdrawn past opening 176 so that flexible wire 178 releases and structure 120 is free from delivery catheter 150.

Preferably coils 140 and 146 and mesh 128 comprise a biocompatible shape memory alloy or polymer such as nitinol, so that the released structure will assume a non-linear, preferably helical or irregular, shape.

It should be appreciated that in the aspect of the invention shown in FIG. 14 the implant is still connected to the delivery “system” via connecting number 178. This is important because the implant can in this partially delivered condition be maneuvered within the patient to either reposition the implant to optimize placement allowing for a controlled delivery, or even to withdraw or retrieve the implant altogether.

In the delivery system shown in FIG. 16, the delivery of an Ultra Soft implant according to the invention is shown. Implant 190 comprises filaments 192 and 194 that form knots 196. Implant 190 has a distal section 200 that comprises a preferably radiopaque coil with helical memory 202 having a proximal washer 204. A proximal section 206 of implant 190 comprises a preferably radiopaque coil with helical memory 208.

Implant 190 is positioned coaxially within a catheter 210, preferably a microcatheter for cranial access and embolism. A pusher sheath 212 has a distal portion 216 with an opening 218. Filaments 192 and 194 form a loop 220 that extends through coil 208 and opening 218 to engage a core wire or mandrel 224. Core wire 224 has a radiopaque distal tip 226. Knots 196 have regularly spaced loops 230 that engage core wire 224.

Core wire 224 has two functions: First, core wire 224 is to provide support to the implant 190 during distal advancement to prevent buckling, due to the nature of the soft material. Core wire 224 distal tip 226 is compressed against distal washer 204 to keep implant 190 at the required tension during distal advancement to the distal part of catheter 210.

Once the distal tip 200 of implant 190 is advanced to the distal tip of catheter 210, core wire 224 is retracted back into coaxial pusher sheath 212 for a few centimeters, for example, from about 2 to about 5 cm., and the core wire 224/sheath 212 assembly is then used to push only implant 190 out of catheter 210 and into an aneurysm (not shown). For implants longer than 5 cm, this process is repeated until the entire length of implant 190 is delivered to the aneurysm. Core wire 224 must always remain within the catheter and be gradually retracted back into the pusher sheath 212 until the entire implant 190 is out of catheter 210 and ready for controlled detachment.

Controlled detachment is the second function of core wire 224. When implant 190 is ready to be detached, core wire 224 is retracted proximally to the extent of opening 218 to release loop 220, whereby implant 190 will separate instantly from the delivery system.

The purpose of proximal coil 208 and distal coil 202 is to provide safe/soft beginning and end of implant deployment into delicate vasculature of the aneurysm wall. The coils preferably have a helical memory, at least one 360° loop, to start folding the implant, as compared to straight penetration deployment. Also, the coils provide excellent radiopaque visibility during initial placement to anchor the implant within the aneurysm sac to prevent migration, by selecting optimal shape memory diameter of the coils to anchor within the diameter of the sac. The coil loop diameter must be larger than the neck/opening of the aneurysm to prevent migration.

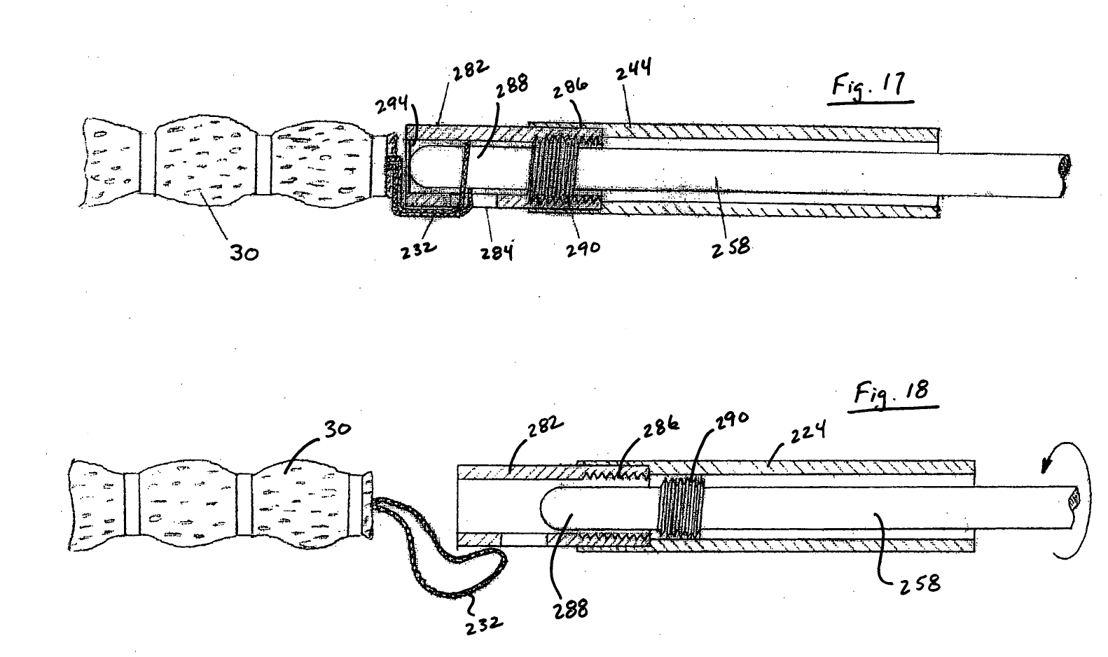

A detail of the connection between the proximal end 234 of implant 30 and the distal end member 282 of pusher member 244 is shown in FIGS. 17 and 18 and describes the suture loop mechanical detachment system used to detach the different implants according to the invention, including Ultra Soft, Soft, and Stiff implants. Distal end member 282 comprises a lateral opening 284 to receive loop 232 from implant 30 and threading 286. The distal end 288 of wire 258 has reciprocal threads 290 that engage threading 286. In the position shown in FIG. 17, the distal end 288 of wire 258 is adjacent to the internal end surface 294 of distal end 282, to trap loop 232. When wire 258 is rotated to cause wire 258 to disengage from threading 286, loop 232 disengages from wire 258 and pusher member 244 and releases implant 30. Also, preferably distal end member 282 comprises radiopaque material such as platinum to assist an operator during delivery. For example, distal end member 282 could comprise a section of platinum hypotube. More preferably, the distal end 288 of wire 258 is also radiopaque, which assists the operator during the procedure. When distal end 228 and distal end 222 are engaged, there will be a single spot under fluoroscopy; however, when distal end 288 and distal end 282 disengage, and release the loop from the implant, there will then be two separate spots under fluoroscopy to signify that release.

Advancing through the microcatheter 270 provides controlled delivery or retraction of implant 30 into the aneurysm cavity with the pusher member 244 until desired positioning of implant 30 is accomplished. Due to the nature of the implant material, the implant fills the aneurysm cavity like a liquid complying with the geometry of the cavity. Continuous flush or pump of hydraulically pressurized solution such as saline solution is applied via microcatheter through the microcatheter side arm at the proximal end to support or drive the advancement of the implant through the catheter lumen. Dependent upon the size of the aneurysm, single or multiple implants may be necessary to achieve total occlusion. The packing density, that is, the ratio of volume of embolic material to volume of the aneurysm sac, ranges from at least about 10% to at least about 200%. Implant 30 can be retracted, before it is detached, and repositioned for precise, controlled deployment and delivery.

Implant 30 is not self-supporting and has no predetermined shape. It conforms significantly better to the geometry of the aneurysm than other implants due to the formation of a light, non-traumatic member filling the cavity like a fluid such as a highly viscous liquid. Because of this important feature the implant material will provide permanent stability of the desired total occlusion. An additional important feature of implant 30 is that it provides excellent tissue ingrowth to seal the aneurysm cavity from the parental artery. There is superior tissue ingrowth due to the porous nature of the reticulated matrix enhanced by structural reticulation created by plication/folding within the aneurysm. Also, plication enhances conformal space filling that eliminates device compaction and recanalization.

Some materials suitable for fabrication of the implants according to the invention will now be described. Implants useful in this invention or a suitable hydrophobic scaffold comprise a reticulated or substantially reticulated polymeric matrix formed of a biodurable polymer that is elastomeric. In one embodiment, polymeric matrix formed of a biodurable polymer is resiliently-compressible so as to regain its shape after being subjected to any compression during delivery to a biological site such as vascular malformations described here. The structure, morphology and properties of the elastomeric matrices of this invention can be engineered or tailored over a wide range of performance by varying the starting materials and/or the processing conditions for different functional or therapeutic uses.

The inventive implantable device is reticulated, i.e., comprises an interconnected network of pores and channels and voids that provides fluid permeability throughout the implantable device and permits cellular and tissue ingrowth and proliferation into the interior of the implantable device. The inventive implantable device is reticulated, i.e., comprises an interconnected and/or inter-communicating network of pores and channels and voids that provides fluid permeability throughout the implantable device and permits cellular and tissue ingrowth and proliferation into the interior of the implantable device. The inventive implantable device is reticulated, i.e., comprises an interconnected and/or inter-communicating network of pores and/or voids and/or channels that provides fluid permeability throughout the implantable device and permits cellular and tissue ingrowth and proliferation into the interior of the implantable device. The biodurable elastomeric matrix or material is considered to be reticulated because its microstructure or the interior structure comprises inter-connected and inter-communicating pores and/or voids bounded by configuration of the struts and intersections that constitute the solid structure. The continuous interconnected void phase is the principle feature of a reticulated structure.

Preferred scaffold materials for the implants have a reticulated structure with sufficient and required liquid permeability and thus selected to permit blood, or other appropriate bodily fluid, and cells and tissues to access interior surfaces of the implants. This happens due to the presence of inter-connected and inter-communicating, reticulated open pores and/or voids and/or channels that form fluid passageways or fluid permeability providing fluid access all through.

In another embodiment the inventive implantable device is only partially reticulated. Thus it contains some segments that are reticulated, i.e., comprises an interconnected network of pores and channels and voids that provides fluid permeability throughout the implantable device and permits cellular and tissue ingrowth and proliferation into the interior of the implantable device. However, it also contains sections that are not reticulated. The inventive implantable device, in another embodiment is partially reticulated, i.e., comprises segments that are interconnected and/or inter-communicating network of pores and channels and voids that provides fluid permeability throughout the implantable device and permits cellular and tissue ingrowth and proliferation into the interior of the implantable device. In this case, one reticulated segment may be separated from another reticulated segment by sections of unreticulated segments.

In another embodiment the inventive implantable device is not necessarily reticulated. It may or may not contain pores and channels and voids. It may or may not contain pores and channels and voids that are interconnected and/or inter-communicating. However, after the device is delivered and the device fills the sac in a way that conforms substantially to the internal shape and volume of the sac, the spaces between the different segments of the device can form at least a partially interconnected and partially inter-communicating space or passage created by plication/folding of the device within the aneurysm. These partially interconnected and partially inter-communicating space or passage can also be created by a single device or by crossing or intersections of multiple devices. This partially interconnected and partially inter-communicating space or passage, can be termed as structural reticulation, and provides fluid permeability throughout the implantable device and permits cellular and tissue ingrowth and proliferation into the interior of the implantable device. In general polymeric matrix, which is preferably biodurable, elastomeric, and reticulated, together with the one or more structural filaments embedded in or incorporated into the matrix, forms an embodiment of the implant of the present invention. However, in the case discussed in this embodiment involving an implantable device or matrix that is not necessarily reticulated, may or may not contain pores and channels and voids and may or may not contain pores and channels and voids that are interconnected and/or inter-communicating, the present invention also teaches that one or more structural filaments need not be embedded in or incorporated into the matrix. It is important to note that in accordance with one of the preferred embodiments of this invention, the column strength or rigidity or biomechanical integrity device or devices of this invention can still be engineered and controlled to facilitate delivery for their advancement through a tortuous catheter or microcatheter and at the same time not make the devices too stiff or too rigid that they are unable to still fold and pack in order to provide a superior packing or filling of the aneurysm on delivery to the aneurysm site.

In another embodiment of the invention the matrix materials for fabricating implants according to the invention are at least partially hydrophobic reticulated, elastomeric polymeric matrix. The materials are flexible and resilient in recovery, so that the implants are also compressible materials enabling the implants to be compressed and, once the compressive force is released, to then recover to, or toward, substantially their original size and shape. For example, an implant can be compressed from a relaxed configuration or a size and shape to a compressed size and shape under ambient conditions, e.g., at 25° C. to fit into the introducer instrument for insertion into the vascular malformations (such as an aneurysm sac or endoloeak nexus within the sac). Alternatively, an implant may be supplied to the medical practitioner performing the implantation operation, in a compressed configuration, for example, contained in a package, preferably a sterile package. The resiliency of the elastomeric matrix that is used to fabricate the implant causes it to recover to a working size and configuration in situ, at the implantation site, after being released from its compressed state within the introducer instrument. The working size and shape or configuration can be substantially similar to original size and shape after the in situ recovery.

In another embodiment, the matrix materials for fabricating implants according to the invention are at least partially hydrophobic partially reticulated, polymeric matrix. These materials are flexible and resilient in recovery, so that the implants are partially compressible materials or non-compressible materials enabling the implants to be slightly compressed or not at all compressed during delivery through a catheter and, once they are released from the catheter to conform substantially to the internal shape and volume of the aneurysm sac. In yet another embodiment, the materials are partially reticulated or not reticulated and the polymeric matrix for fabricating implants according to the invention are still flexible and resilient in recovery, so that the implants are somewhat compressible materials or non-compressible materials enabling the implants to be slightly compressed or not at all compressed during delivery through a catheter but once they are released from the catheter to conform substantially to the internal shape and volume of the aneurysm sac. The phenomenon of conforming substantially to the internal shape and volume of the aneurysm sac will cause more effective healing of the aneurysm.

In another embodiment, the materials are at least partially hydrophobic partially reticulated, polymeric matrix for fabricating implants according to the invention are visoelastic without being partially or substantially elastomeric. If the device or the material from which the device is made is not flexible enough or it is too stiff, the device will not be deliverable through the catheter or will not easily pushable through the tortuous contours of the catheters in the human anatomy and may even clog the catheter. The flexibility necessary for delivery through tortuous contours of the catheters placed in the human anatomy and/or for conforming substantially to the internal shape and volume of the sac may come from the inherent flexibility or lower mechancial properties of the material and in one embodiment can be engineered from relatively stiffer materials by the creation of voids and defects in the matrix. Again, when implants according to the invention are visoelastic without being partially or substantially elastomeric, the present invention also teaches that one or more structural filaments need not be embedded in or incorporated into the matrix.

Preferred scaffolds are reticulated elastomeric polymeric materials having sufficient structural integrity and durability to endure the intended biological environment, for the intended period of implantation. In another embodiment, scaffolds of partially reticulated, substantially reticulated or non-reticulated elastomeric polymeric materials having sufficient structural integrity and durability to endure the intended biological environment, for the intended period of implantation. In another embodiment, scaffolds of reticulated, partially reticulated, substantially reticulated or non-reticulated viscoelastic polymeric materials having sufficient structural integrity and durability to endure the intended biological environment, for the intended period of implantation. For structure and durability, at least partially hydrophobic polymeric scaffold materials are preferred although other materials may be employed if they meet the requirements described herein. Useful materials are preferably elastomeric in that they can be compressed and can resiliently recover to substantially to the pre-compression state. Alternative to reticulated polymeric materials, other materials with pores or networks of pores that may or may not be interconnected that permit biological fluids to have ready access throughout the interior of an implant may be employed, for example, woven or nonwoven fabrics or networked composites of microstructural elements of various forms.

A partially hydrophobic scaffold is preferably constructed of a material selected to be sufficiently biodurable, for the intended period of implantation that the implant will not lose its structural integrity during the implantation time in a biological environment. The biodurable elastomeric matrices forming the scaffold do not exhibit significant symptoms of breakdown, degradation, erosion, or significant deterioration of mechanical properties relevant to their use when exposed to biological environments and/or bodily stresses for periods of time commensurate with the use of the implantable device. In one embodiment, the desired period of exposure is to be understood to be at least 29 days, preferably several weeks and most preferably 2 to 5 years or more. This measure is intended to avoid scaffold materials that may decompose or degrade into fragments, for example, fragments that could have undesirable effects such as causing an unwanted tissue response.

The void phase, preferably continuous and interconnected, of the reticulated polymeric matrix that is used to fabricate the implant of this invention may comprise as little as 50% by volume of the elastomeric matrix, referring to the volume provided by the interstitial spaces of elastomeric matrix before any optional interior pore surface coating or layering is applied. In one embodiment, the volume of void phase as just defined, is from about 20% to about 50% of the volume of elastomeric matrix. In another embodiment, the volume of void phase as just defined, is from about 50% to about 70% of the volume of elastomeric matrix. In another embodiment, the volume of void phase as just defined, is from about 70% to about 99% of the volume of elastomeric matrix. In another embodiment, the volume of void phase is from about 80% to about 98% of the volume of elastomeric matrix. In another embodiment, the volume of void phase is from about 90% to about 98% of the volume of elastomeric matrix. In another embodiment, the void phase is not continuous and interconnected in one or several contiguous segments of the device or is not continuous throughout the entire device.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. For those skilled in the art, one can routinely estimate the pore frequency from the average cell diameter in microns.

In one embodiment relating to vascular malformation applications and the like, to encourage cellular ingrowth and proliferation and to provide adequate fluid permeability, the average diameter or other largest transverse dimension of pores is at least about 50 μm. In another embodiment, the average diameter or other largest transverse dimension of pores is at least about 100 μm. In another embodiment, the average diameter or other largest transverse dimension of pores is at least about 150 μm. In another embodiment, the average diameter or other largest transverse dimension of pores is at least about 250 μm. In another embodiment, the average diameter or other largest transverse dimension of pores is greater than about 250 μm. In another embodiment, the average diameter or other largest transverse dimension of pores is greater than 250 μm. In another embodiment, the average diameter or other largest transverse dimension of pores is at least about 275 μm. In another embodiment, the average diameter or other largest transverse dimension of pores is greater than about 275 μm. In another embodiment, the average diameter or other largest transverse dimension of pores is greater than 275 μm. In another embodiment, the average diameter or other largest transverse dimension of pores is at least about 300 μm. In another embodiment, the average diameter or other largest transverse dimension of pores is greater than about 300 μm. In another embodiment, the average diameter or other largest transverse dimension of pores is greater than 300 μm.

In another embodiment, the average diameter or other largest transverse dimension of pores is not greater than about 700 μm. In another embodiment, the average diameter or other largest transverse dimension of pores is not greater than about 600 μm. In another embodiment, the average diameter or other largest transverse dimension of pores is not greater than about 500 μm.

In one embodiment, the reticulated polymeric matrix that is used to fabricate the implants of this invention has any suitable bulk density, also known as specific gravity, consistent with its other properties. For example, in one embodiment, the bulk density may be from about 0.005 to about 0.15 g/cc (from about 0.31 to about 9.4 lb/ft³), preferably from about 0.015 to about 0.104 g/cc (from about 0.93 to about 6.5 lb/ft³) and most preferably from about 0.024 to about 0.080 g/cc (from about 1.5 to about 5.0 lb/ft³).

The polymeric matrix has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix. Thus, for example, in one embodiment, the reticulated polymeric matrix that is used to fabricate the implants of this invention may have a tensile strength of from about 700 to about 87,500 kg/m² (from about 1 to about 125 psi). In another embodiment, elastomeric matrix may have a tensile strength of from about 3500 to about 52,500 kg/m² (from about 5 to about 75 psi). Sufficient ultimate tensile elongation is also desirable. For example, in another embodiment, reticulated elastomeric matrix has an ultimate tensile elongation of at least about 50% to at least about 500%. In yet another embodiment, reticulated elastomeric matrix has an ultimate tensile elongation of at least 75% to at least about 300%.

One embodiment for use in the practice of the invention is reticulated or at least partially reticulated or substantially reticulated or non-reticulated elastomeric implant which is sufficiently flexible, i.e it can be delivered from a relaxed configuration for delivery via a delivery-device, e.g., catheter, endoscope, syringe, cystoscope, trocar or other suitable introducer instrument, for delivery in vitro and, thereafter, into a second, working configuration in situ, preferably without compressing the device during delivery through a delivery device of the invention. In another embodiment for use in the practice of the invention is a reticulated or at least partially reticulated or substantially reticulated elastomeric implant which is sufficiently resilient, i.e., resiliently-compressible, to enable it to be initially compressed under ambient conditions, e.g., at 25° C., from a relaxed configuration to a first, compact configuration for delivery via a delivery-device, e.g., catheter, endoscope, syringe, cystoscope, trocar or other suitable introducer instrument, for delivery in vitro and, thereafter, to expand to a second, working configuration in situ. In one embodiment, the device can be delivered without being compacted during delivery or it can be compacted less than 5% of an original dimension during delivery. Furthermore, in another embodiment, an elastomeric matrix has the herein described resilient-compressibility after being compressed about 5-95% of an original dimension. In another embodiment, an elastomeric matrix has the herein described resilient-compressibility after being compressed about 10-90% of an original dimension. As used herein, elastomeric implant has “resilient-compressibility”, i.e., is “resiliently-compressible”, when the second, working configuration, in vitro, is at least about 50% of the size of the relaxed configuration in at least one dimension. In another embodiment, the resilient-compressibility of elastomeric implant is such that the second, working configuration, in vitro, is at least about 80% of the size of the relaxed configuration in at least one dimension. In another embodiment, the resilient-compressibility of elastomeric implant is such that the second, working configuration, in vitro, is at least about 90% of the size of the relaxed configuration in at least one dimension. In another embodiment, the resilient-compressibility of elastomeric implant is such that the second, working configuration, in vitro, is at least about 97% of the size of the relaxed configuration in at least one dimension.

In one embodiment, the device can be delivered without being compacted during delivery or it can be compacted less than 5% of an original volume during delivery. In another embodiment, an elastomeric matrix has the herein described resilient-compressibility after being compressed about 5-95% of its original volume. In another embodiment, an elastomeric matrix has the herein described resilient-compressibility after being compressed about 10-90% of its original volume. As used herein, “volume” is the volume swept-out by the outermost three-dimensional contour of the elastomeric matrix. In another embodiment, the resilient-compressibility of elastomeric implant is such that the second, working configuration, in vivo, is at least about 50% of the volume occupied by the relaxed configuration. In another embodiment, the resilient-compressibility of elastomeric implant is such that the second, working configuration, in vivo, is at least about 80% of the volume occupied by the relaxed configuration. In another embodiment, the resilient-compressibility of elastomeric implant is such that the second, working configuration, in vivo, is at least about 90% of the volume occupied by the relaxed configuration. In another embodiment, the resilient-compressibility of elastomeric implant is such that the second, working configuration, in vivo, occupies at least about 97% of the of volume occupied by the elastomeric matrix in its relaxed configuration.

Without being bound by any particular theory, it is believed that the absence or substantial absence of cell walls in reticulated implants when compressed to very high degree will allow them to demonstrate resilient recovery in shorter time (such as recovery time of under 15 seconds when compressed to 75% of their relaxed configuration for 10 minutes and recovery time of under 35 seconds when compressed to 90% of their relaxed configuration for 10 minutes) as compared to un-reticulated porous foams.

In one embodiment, reticulated elastomeric matrix that is used to fabricate the implants of this invention has a compressive strength of from about 700 to about 70,000 kg/m² (from about 1 to about 100 psi) at 50% compression strain. In another embodiment, reticulated elastomeric matrix has a compressive strength of from about 1,225 to about 105,000 kg/m² (from about 1.75 to about 150 psi) at 75% compression strain.

In another embodiment, reticulated elastomeric matrix that is used to fabricate the implants of this invention has a compression set, when compressed to 50% of its thickness at about 25° C., of not more than about 30%. In another embodiment, elastomeric matrix has a compression set of not more than about 20%. In another embodiment, elastomeric matrix has a compression set of not more than about 10%. In another embodiment, elastomeric matrix has a compression set of not more than about 5%.

In another embodiment, reticulated elastomeric matrix that is used to fabricate the implants of this invention has a tear strength, of from about 0.18 to about 1.78 kg/linear cm (from about 1 to about 10 lbs/linear inch).

In another embodiment of the invention the reticulated elastomeric matrix that is used to fabricate the implant can be readily permeable to liquids, permitting flow of liquids, including blood, through the composite device of the invention. The water permeability (Darcy) of the reticulated elastomeric matrix is from about 50 to about 500 (from about 0.204 to 2.04 lit/min/psi/cm/sq.cm for flow rate of water through the matrix), preferably from about 100 to about 300 (0.408 to 1.224 lit/min/psi/cm/sq.cm for flow rate of water through the matrix). In contrast, permeability (Darcy) of the unreticulated elastomeric matrix is below about 1. In another embodiment, the permeability (Darcy) of the unretriculated elastomeric matrix is below about 5.

In general, suitable biodurable reticulated elastomeric partially hydrophobic polymeric matrix that is used to fabricate the implant of this invention or for use as scaffold material for the implant in the practice of the present invention, in one embodiment sufficiently well characterized, comprise elastomers that have or can be formulated with the desirable mechanical properties described in the present specification and have a chemistry favorable to biodurability such that they provide a reasonable expectation of adequate biodurability.

Various biodurable reticulated hydrophobic polyurethane materials are suitable for this purpose. In one embodiment, structural materials for the inventive reticulated elastomers are synthetic polymers, especially, but not exclusively, elastomeric polymers that are resistant to biological degradation, for example, polycarbonate polyurethane-urea, polycarbonate polyurea-urethane, polycarbonate polyurethane, polycarbonate polysiloxane polyurethane, and polysiloxane polyurethane, and the like. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The invention can employ, for implanting, a biodurable reticulatable elastomeric partially hydrophobic polymeric scaffold material or matrix for fabricating the implant or a material. More particularly, in one embodiment, the invention provides a biodurable elastomeric polyurethane scaffold material or matrix which is made by synthesizing the scaffold material or matrix preferably from a polycarbonate polyol component and an isocyanate component by polymerization, cross-linking and foaming, thereby forming pores, followed by reticulation of the porous material to provide a biodurable reticulated elastomeric product with inter-connected and/or inter-communicating pores and channels. The product is designated as a polycarbonate polyurethane, being a polymer comprising urethane groups formed from, e.g., the hydroxyl groups of the polycarbonate polyol component and the isocyanate groups of the isocyanate component. In another embodiment, the invention provides a biodurable elastomeric polyurethane scaffold material or matrix which is made by synthesizing the scaffold material or matrix preferably from a polycarbonate polyol component and an isocyanate component by polymerization, cross-linking and foaming, thereby forming pores, and using water as a blowing agent and/or foaming agent during the synthesis, followed by reticulation of the porous material to provide a biodurable reticulated elastomeric product with inter-connected and/or inter-communicating pores and channels. This product is designated as a polycarbonate polyurethane-urea or polycarbonate polyurea-urethane, being a polymer comprising urethane groups formed from, e.g., the hydroxyl groups of the polycarbonate polyol component and the isocyanate groups of the isocyanate component and also comprising urea groups formed from reaction of water with the isocyanate groups. In all of these embodiments, the process employs controlled chemistry to provide a reticulated elastomeric matrix or product with good biodurability characteristics. The matrix or product employing chemistry that avoids biologically undesirable or nocuous constituents therein.

In one embodiment, the starting material for synthesizing the biodurable reticulated elastomeric partially hydrophobic polymeric matrix contains at least one polyol component to provide the so-called soft segement. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. In one embodiment, this soft segment polyol is terminated with hydroxyl groups, either primary or secondary. Exemplary polyols can comprise, on the average, from about 2 to about 5 hydroxyl groups per molecule. In one embodiment, as one starting material, the process employs a difunctional polyol component in which the hydroxyl group functionality of the diol is about 2. In another embodiment, the soft segment is composed of a polyol component that is generally of a relatively low molecular weight, typically from about 500 to about 6,000 daltons and preferably between 1000 to 2500 Daltons. Examples of suitable polyol components include but not limited to polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol, polysiloxane polyol and copolymers and mixtures thereof.

In one embodiment, the starting material for synthesizing the biodurable reticulated elastomeric partially hydrophobic polymeric matrix contains at least one isocyanate component and, optionally, at least one chain extender component to provide the so-called “hard segment”. In another embodiment, the starting material for synthesizing the biodurable reticulated elastomeric partially hydrophobic polymeric matrix contains at least one isocyanate component. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, crosslinker and/or water. In another embodiment, the average number of isocyanate groups per molecule in the isocyanate component is about 2. In another embodiment, the average number of isocyanate groups per molecule in the isocyanate component is greater than about 2 is greater than 2.

Exemplary diisocyanates include aliphatic diisocyanates, isocyanates comprising aromatic groups, the so-called “aromatic diisocyanates”, and mixtures thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H12 MDI”), and mixtures thereof Aromatic diisocyanates include p-phenylene diisocyanate, 4,4′-diphenylmethane diisocyanate (“4,4′-MDI”), 2,4′-diphenylmethane diisocyanate (“2,4′-MDI”), polymeric MDI, and mixtures thereof. Examples of optional chain extenders include diols, diamines, alkanol amines or a mixture thereof.

In another embodiment, a small quantity of an optional ingredient, such as a multi-functional hydroxyl compound or other cross-linker having a functionality greater than 2, is present to allow crosslinking and/or to achieve a stable foam, i.e., a foam that does not collapse to become non-foamlike. Alternatively, or in addition, polyfunctional adducts of aliphatic and cycloaliphatic isocyanates can be used to impart cross-linking in combination with aromatic diisocyanates. Alternatively, or in addition, polyfunctional adducts of aliphatic and cycloaliphatic isocyanates can be used to impart cross-linking in combination with aliphatic diisocyanates. The presence of these components and adducts with functionality higher than 2 in the hard segment component allows for cross-linking to occur.

In another embodiment, a small quantity of an optional ingredient such as 1,4 butane diol is present as a chain extender.

In one embodiment, the starting material for synthesizing the biodurable reticulated elastomeric partially hydrophobic polymeric matrix contains at least one blowing agent such as water. Other exemplary blowing agents include the physical blowing agents, e.g., volatile organic chemicals such as hydrocarbons, ethanol and acetone, and various fluorocarbons, hydrofluorocarbons, chlorofluorocarbons, and hydrochlorofluorocarbons. In another embodiment, the hard segments also contain a urea component formed during foaming reaction with water. In another embodiment, the reaction of water with an isocyanate group yields carbon dioxide, which serves as a blowing agent. The amount of blowing agent, e.g., water, is adjusted to obtain different densities of non-reticulated foams. A reduced amount of blowing agent such as water may reduce the number of urea linkages in the material.

In another embodiment, the starting material of the biodurable reticulated elastomeric partially hydrophobic polymeric matrix is a commercial polyurethane polymers are linear, not crosslinked, polymers, therefore, they are soluble, can be melted, readily analyzable and readily characterizable. In this embodiment, the starting polymer provides good biodurability characteristics. The reticulated elastomeric matrix is produced by taking a solution of the commercial polymer such as polyurethane and optionally charging it into a mold that has been fabricated with surfaces defining a microstructural configuration for the final implant or scaffold, solidifying the polymeric material and removing the sacrificial mold by melting, dissolving or subliming-away the sacrificial mold. The matrix or product employing a foaming process that avoids biologically undesirable or nocuous constituents therein. In another embodiment, the reticulated elastomeric matrix is produced by taking a solution of the commercial polymer such as polyurethane and charging it into a mold, and lyophilizing, i.e., subliming-away and removing the solvent.

Of particular interest are thermoplastic elastomers such as polyurethanes whose chemistry is associated with good biodurability properties, for example. In one embodiment, such thermoplastic polyurethane elastomers include polycarbonate polyurethanes, polysiloxane polyurethanes, polyurethanes with so-called “mixed” soft segments, and mixtures thereof. Mixed soft segment polyurethanes are known to those skilled in the art and include, e.g., polycarbonate-polysiloxane polyurethanes. In another embodiment, the thermoplastic polyurethane elastomer comprises at least one diusocyanate in the isocyanate component, at least one chain extender and at least one diol, and may be formed from any combination of the diisocyanates, difunctional chain extenders and diols described in detail above. Some suitable thermoplastic polyurethanes for practicing the invention, in one embodiment suitably characterized as described herein, include: polyurethanes with mixed soft segments comprising polysiloxane together with a polycarbonate component.

In one embodiment, the weight average molecular weight of the thermoplastic elastomer is from about 30,000 to about 500,000 Daltons. In another embodiment, the weight average molecular weight of the thermoplastic elastomer is from about 50,000 to about 250,000 Daltons.

Some commercially-available thermoplastic elastomers suitable for use in practicing the present invention include the line of polycarbonate polyurethanes supplied under the trademark BIONATE® by The Polymer Technology Group Inc. (Berkeley, Calif.). For example, the very well-characterized grades of polycarbonate polyurethane polymer BIONATE® 80A, 55 and 90 are soluble in THF, DMF, DMAT, DMSO, or a mixture of two or more thereof, processable, reportedly have good mechanical properties, lack cytotoxicity, lack mutagenicity, lack carcinogenicity and are non-hemolytic. Another commercially-available elastomer suitable for use in practicing the present invention is the CHRONOFLEX® C line of biodurable medical grade polycarbonate aromatic polyurethane thermoplastic elastomers available from CardioTech International, Inc. (Woburn, Mass.).

In another embodiment, the starting material of the biodurable reticulated, substantially reticulated, partially reticulated or non-reticulated elastomeric partially hydrophobic polymeric matrix is a commercial viscoelastic thermoplastic including both semi-crystalline and amorphous materials, polymers, therefore, they are soluble, can be melted, readily analyzable and readily characterizable. In another embodiment, In this embodiment, the starting polymer provides good biodurability characteristics. Exemplary viscoelastic thermoplastic, although not limited only to the following list, includes suitable biocompatible polymers include polyamides, polyolefins (e.g., polypropylene, polyethylene), nonabsorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone and blends thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules and blends thereof. For the purpose of this invention aliphatic polyesters include polymers and copolymers of lactide (which includes lactic acid d-, 1- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and blends thereof.

Biocompatible polymers further include film-forming biodurable polymers with relatively low chronic tissue response, such as polyurethanes, silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from crosslinked polyvinyl pyrrolidinone and polyesters. Other polymers, of course, can also be used as the biocompatible polymer provided that they can be dissolved, cured or polymerized. Such polymers and copolymers include polyolefins, polyisobutylene and ethylene-α-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with α-olefins, such as etheylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e.g., carboxymethyl cellulose and hydoxyalkyl celluloses); and mixtures thereof. For the purpose of this invention, polyamides include polyamides of the general forms:
—N(H)—(CH₂)ₙ—C(O)— and —N(H)—(CH₂)ₓ—N(H)—C(O)—(CH₂)_{y}—C(O)—,
where n is an integer from about 4 to about 13; x is an integer from about 4 to about 12; and y is an integer from about 4 to about 16. It is, of course, to be understood that the listings of materials above are illustrative but not limiting.

In another embodiment the starting material of the biodurable reticulated, substantially reticulated, partially reticulated or non-reticulated partially hydrophobic polymeric matrix are viscoelastic crosslinked and are thermosets. In some cases the viscoelastic crosslinked are elastomeric.

There are various alternative methods of making the inventive devices from the list of suitable viscoelastic biocompatible thermoplastic and crosslinked or thermoset materials and some exemplary ones include extrusion, co-extrusion, extrusion coating, solution coating, injection molding, co-injection molding, film blowing, compression molding, thermoforming, gas assisted melt extrusion with appropriate pressure release to create a porous structure, various short and long fiber composite technologies including injection molding, extrusion fiber impregnation, mesh impregnation, extrusion and injection molding of leachable fillers such as salt and sugar followed by removal of the fillers by solvent, extraction or washing, etc. While the preceding list can be considered as primary processing steps, secondary processing steps such as shaping, forming, hole punching, die punching, annealing, solid state drawing, drawing at elevated temperatures, orientation, etc. can also be used to form the inventive device from suitable viscoelastic biocompatible thermoplastic and crosslinked or thermoset materials.

Other possible embodiments of the materials used to fabricate the implants of this invention are described in co-pending, commonly assigned U.S. patent applications Ser. No. 10/749,742, filed Dec. 30, 2003, titled “Reticulated Elastomeric Matrices, Their Manufacture and Use in Implantable Devices”, Ser. No. 10/848,624, filed May 17, 2004, titled “Reticulated Elastomeric Matrices, Their Manufacture and Use In Implantable Devices”, and Ser. No. 10/990,982, filed Jul. 27, 2004, titled “Endovascular Treatment Devices and Methods”, each of which is incorporated herein by reference in its entirety.

If desired, the reticulated elastomeric implants or implants for packing the aneurysm sac or for other vascular occlusion can be rendered radiopaque to allow for visualization of the implants in situ by the clinician during and after the procedure, employing radioimaging. Any suitable radiopaque agent that can be covalently bound, adhered or otherwise attached to the reticulated polymeric implants may be employed including without limitation, tantalum and barium sulfate. In addition to incorporating radiopaque agents such as tantalum into the implant material itself, a further embodiment of the invention encompasses the use of radiopaque metallic components to impart radiopacity to the implant. For example, thin filaments comprised of metals with shape memory properties such as platinum can be embedded into the implant and may be in the form of a straight or curved wire, helical or coil-like structure, umbrella structure, or other structure generally known to those skilled in the art. In another embodiment, thin filaments comprised of metals do not need to possess shape memory properties. Exemplary filaments include platinum or nitinol. In another embodiment, the structural fiber or components of the of structural fiber the inventive device is at east partially radiopaque. In another embodiment, radiopaque markers that are preferably metallic can be crimped at regular intervals along the device. Alternatively, a metallic frame around the implant may also be used to impart radiopacity. The metallic frame may be in the form of a tubular structure similar to a stent, a helical or coil-like structure, an umbrella structure, or other structure generally known to those skilled in the art. Attachment of radiopaque metallic components to the implant can be accomplished by means including but not limited to chemical bonding or adhesion, suturing, pressure fitting, compression fitting, and other physical methods.

Some optional embodiments of the invention comprise apparatus or devices and treatment methods employing biodurable at least partially reticulated elastomeric implants or substantially reticulated elastomeric implants into which biologically active agents are incorporated for the matrix to be used for controlled release of pharmaceutically-active agents, such as a drug, and for other medical applications. In another embodiment, the invention comprise apparatus or devices and treatment methods employing biodurable non-reticulated implants into which biologically active agents are incorporated for the matrix to be used for controlled release of pharmaceutically-active agents, such as a drug, and for other medical applications. Any suitable agents may be employed as will be apparent to those skilled in the art, including, for example, but without limitation thrombogenic agents, e.g., thrombin, anti-inflammatory agents, and other therapeutic agents that may be used for the treatment of abdominal aortic aneurysms. The invention includes embodiments wherein the reticulated elastomeric material of the implants is employed as a drug delivery platform for localized administration of biologically active agents into the aneurysm sac. Such materials may optionally be secured to the interior surfaces of elastomeric matrix directly or through a coating. In one embodiment of the invention the controllable characteristics of the implants are selected to promote a constant rate of drug release during the intended period of implantation.

The implants, with reticulated structure with sufficient and required liquid permeability, permit blood or another appropriate bodily fluid to access interior surfaces of the implants, which surfaces are optionally are drug-bearing. This happens due to the presence of inter-connected, reticulated open pores that form fluid passageways or fluid permeability providing fluid access all through and to the interior of the matrix for elution of pharmaceutically-active agents, e.g., a drug, or other biologically useful materials.

In a further embodiment of the invention, the pores of biodurable reticulated elastomeric matrix that are used to fabricate the implants of this invention are coated or filled with a cellular ingrowth promoter. In another embodiment, the promoter can be foamed. In another embodiment, the promoter can be present as a film. The promoter can be a biodegradable material to promote cellular invasion of pores biodurable reticulated elastomeric matrix that are used to fabricate the implants of this invention in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of the biodurable reticulated elastomeric matrix that are used to fabricate the implants of this invention is coated or impregnated, as described in the previous section but substituting the promoter for the biocompatible polymer or adding the promoter to the biocompatible polymer, to encourage cellular ingrowth and proliferation.

One possible material for use in the present invention comprises a resiliently compressible composite polyurethane material comprising a hydrophilic foam coated on and throughout the pore surfaces of a hydrophobic foam scaffold. One suitable such material is the composite foam disclosed in co-pending, commonly assigned U.S. patent applications Ser. No. 10/692,055, filed Oct. 22, 2003, Ser. No. 10/749,742, filed Dec. 30, 2003, Ser. No. 10/848,624, filed May 17, 2004, and Ser. No. 10/900,982, filed Jul. 27, 2004, each of which is incorporated herein by reference in its entirety. The hydrophobic foam provides support and resilient compressibility enabling, the desired collapsing of the implant for delivery and reconstitution in situ.

The reticulated biodurable elastomeric and at least partially hydrophilic material can be used to carry a variety of therapeutically useful agents, for example, agents that can aid in the healing of the aneurysm, such as elastin, collagen or other growth factors that will foster fibroblast proliferation and ingrowth into the aneurysm, agents to render the foam implant non-thrombogenic, or inflammatory chemicals to foster scarring of the aneurysm. Furthermore the hydrophilic foam, or other agent immobilizing means, can be used to carry genetic therapies, e.g. for replacement of missing enzymes, to treat atherosclerotic plaques at a local level, and to release agents such as antioxidants to help combat known risk factors of aneurysm.

Pursuant to the present invention it is contemplated that the pore surfaces may employ other means besides a hydrophilic foam to secure desired treatment agents to the hydrophobic foam scaffold.

The agents contained within the implant can provide an inflammatory response within the aneurysm, causing the walls of the aneurysm to scar and thicken. This can be accomplished using any suitable inflammation inducing chemicals, such as sclerosants like sodium tetradecyl sulphate (STS), polyiodinated iodine, hypertonic saline or other hypertonic salt solution. Additionally, the implant can contain factors that will induce fibroblast proliferation, such as growth factors, tumor necrosis factor and cytokines.

The fibers used in the present invention for as a component of reinforcing filaments is polymeric and may be made using a variety of processes that provide fibers with the desired properties (such as modulus, tensile strength, elongation etc.). Those skilled in the art of fiber processing are well versed in the art of extrusion, solution spinning etc. which may be used to provide polymer based fibers. These fibers may be oriented or drawn using conventional process to provide the desired degree of modulus, strength, elongation, etc. Generally, a fiber orientation process is used to improve the properties of the reinforcing fibers. Suitable organic biocompatible polymer that can be used to make the polymeric fibers are well known in the art and include both bioabsorbable and biostable polymers.

For example, bioabsorbable polymeric fiber can be made from a polymer or copolymer or blend containing glycolide, L-lactide, D-lactide, caprolactone, para-dioxanone and/or trimethylene carbonate and combinations thereof.

Suitable organic biocompatible biostable polymeric fiber can be made from include but are not limited to polymers selected from the group consisting of polyesters (such as polyethylene terephthalate and polybutylene terephthalate), polyolefins (such as polyethylene and polypropylene including atactic, isotactic, syndiotactic, and blends thereof as well as, polyisobutylene and ethylene-alphaolefin copolymers), polyamides (such as nylon 4, nylon 6, nylon 66, nylon 610, nylon 11, nylon 12), acrylic polymers and copolymers, polycarbonates, polyurethanes and their copolymers, blends and combinations thereof.

In another embodiment, the fibers used in the present invention for as a component of reinforcing filaments can be made from glass fibers or carbon fiber, the likes of which are commonly used to reinforce polymeric composites.

The fibers used in the present invention for as a component of reinforcing filaments can have a diameter that range from 0.01 mm to 0.40 mm and preferably from 0.02 mm to 0.30 mm. In one embodiment, the fibers used in the present invention for as a component of reinforcing filaments can be any commercially available, non-absorbable polymeric or absorbable suture.

### EXAMPLES

### Example 1

### Fabrication of a Cross-Linked Reticulated Polyurethane Matrix

The aromatic isocyanate RUBINATE 9258 (from Huntsman) was used as the isocyanate component. RUBINATE 9258, which is a liquid at 25° C., contains 4,4′-MDI and 2,4′-MDI and has an isocyanate functionality of about 2.33. A diol, poly(1,6-hexanecarbonate)diol (POLY-CD CD220 from Arch Chemicals) with a molecular weight of about 2,000 Daltons was used as the polyol component and was a solid at 25° C. Distilled water was used as the blowing agent. The blowing catalyst used was the tertiary amine triethylenediamine (33% in dipropylene glycol; DABCO 33LV from Air Products). A silicone-based surfactant was used (TEGOSTAB® BF 2370 from Goldschmidt). A cell-opener was used (ORTEGOL® 501 from Goldschmidt). The viscosity modifier propylene carbonate (from Sigma-Aldrich) was present to reduce the viscosity. The proportions of the components that were used are set forth in the following table:

TABLE 2

| Ingredient | Parts by Weight |
|---|---|
| Polyol Component | 100 |
| Viscosity Modifier | 5.80 |
| Surfactant | 0.66 |
| Cell Opener | 1.00 |
| Isocyanate Component | 47.25 |
| Isocyanate Index | 1.00 |
| Distilled Water | 2.38 |
| Blowing Catalyst | 0.53 |

The polyol component was liquefied at 70° C. in a circulating-air oven, and 100 g thereof was weighed out into a polyethylene cup. 5.8 g of viscosity modifier was added to the polyol component to reduce the viscosity, and the ingredients were mixed at 3100 rpm for 15 seconds with the mixing shaft of a drill mixer to form “Mix-1”. 0.66 g of surfactant was added to Mix-1, and the ingredients were mixed as described above for 15 seconds to form “Mix-2”. Thereafter, 1.00 g of cell opener was added to Mix-2, and the ingredients were mixed as described above for 15 seconds to form “Mix-3”. 47.25 g of isocyanate component were added to Mix-3, and the ingredients were mixed for 60±10 seconds to form “System A”.

2.38 g of distilled water was mixed with 0.53 g of blowing catalyst in a small plastic cup for 60 seconds with a glass rod to form “System B”.

System B was poured into System A as quickly as possible while avoiding spillage. The ingredients were mixed vigorously with the drill mixer as described above for 10 seconds and then poured into a 22.9 cm×20.3 cm×12.7 cm (9 in.×8 in.×5 in.) cardboard box with its inside surfaces covered by aluminum foil. The foaming profile was as follows: 10 seconds mixing time, 17 seconds cream time, and 85 seconds rise time.

Two minutes after the beginning of foaming, i.e., the time when Systems A and B were combined, the foam was placed into a circulating-air oven maintained at 100-105° C. for curing for from about 55 to about 60 minutes. Then, the foam was removed from the oven and cooled for 15 minutes at about 25° C. The skin was removed from each side using a band saw. Thereafter, hand pressure was applied to each side of the foam to open the cell windows. The foam was replaced into the circulating-air oven and postcured at 100-105° C. for an additional four hours.

The average pore diameter of the foam, as determined from optical microscopy observations, was greater than about 275 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 2.81 lbs/ft³ (0.0450 g/cc) was obtained.

Tensile tests were conducted on samples that were cut either parallel to or perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm, and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 29.3 psi (20,630 kg/m²). The elongation to break perpendicular to the direction of foam rise was determined to be 266%.

The measurement of the liquid flow through the material is measured in the following way using a liquid permeability apparatus or Liquid Permeaeter (Porous Materials, Inc., Ithaca, N.Y.). The foam sample was 8.5 mm in thickness and covered a hole 6.6 mm in diameter in the center of a metal plate that was placed at the bottom of the Liquid Permeaeter device filled with water. Thereafter, the air pressure above the sample was increased slowly to extrude the liquid from the sample, and the permeability of water (Darcy) through the foam was determined to be 0.11.

### Example 2

### Reticulation of a Cross-Linked Polyurethane Foam

Reticulation of the foam described in Example 1 was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

The average pore diameter of the reticulated elastomeric matrix, as determined from optical microscopy observations, was greater than about 275 μm. A scanning electron micrograph image of the reticulated elastomeric matrix of this example (not shown here) demonstrated, e.g., the communication and interconnectivity of pores therein.

The density of the reticulated foam was determined as described above in Example 1. A post-reticulation density value of 2.83 lbs/ft³ (0.0453 g/cc) was obtained.

Tensile tests were conducted on reticulated foam samples as described above in Example 1. The average post-reticulation tensile strength perpendicular to the direction of foam rise was determined as about 26.4 psi (18,560 kg/m²). The post-reticulation elongation to break perpendicular to the direction of foam rise was determined to be about 250%. The average post-reticulation tensile strength parallel to the direction of foam rise was determined as about 43.3 psi (30,470 kg/m²). The post-reticulation elongation to break parallel to the direction of foam rise was determined to be about 270%.

Compressive tests were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths at 50% compression, parallel to and perpendicular to the direction of foam rise, were determined to be 1.53 psi (1,080 kg/m²) and 0.95 psi (669 kg/m²), respectively. The post-reticulation compressive strengths at 75% compression, parallel to and perpendicular to the direction of foam rise, were determined to be 3.53 psi (2,485 kg/m²) and 2.02 psi (1,420 kg/m²), respectively. The post-reticulation compression set, determined after subjecting the reticulated sample to 50% compression for 22 hours at 25° C. then releasing the compressive stress, parallel to the direction of foam rise, was determined to be about 4.5%.

The resilient recovery of the reticulated foam was measured by subjecting 1 inch (25.4 mm) diameter and 0.75 inch (19 mm) long foam cylinders to 75% uniaxial compression in their longitudinal direction for 10 or 30 minutes and measuring the time required for recovery to 90% (“t-90%”) and 95% (“t-95%”) of their initial length. The percentage recovery of the initial length after 10 minutes (“r-10”) was also determined. Separate samples were cut and tested with their length direction parallel to and perpendicular to the foam rise direction. The results obtained from an average of two tests are shown in the following table:

TABLE 3

| Time | | | | |
|---|---|---|---|---|
| compressed | Test Sample | t-90% | t-95% | r-10 |
| (min) | Orientation | (sec) | (sec) | (%) |
| 10 | Parallel | 6 | 11 | 100 |
| 10 | Perpendicular | 6 | 23 | 100 |
| 30 | Parallel | 9 | 36 | 99 |
| 30 | Perpendicular | 11 | 52 | 99 |

In contrast, a comparable foam with little to no reticulation typically has t-90 values of grater than about 60-90 seconds after 10 minutes of compression.

The measurement of the liquid flow through the material is measured in the following way using a liquid permeability apparatus or Liquid Permeaeter (Porous Materials, Inc., Ithaca, N.Y.). The foam samples were between 7.0 and 7.7 mm in thickness and covered a hole 8.2 mm in diameter in the center of a metal plate that was placed at the bottom of the Liquid Permeaeter device filled with water. The water was allowed to extrude through the sample under gravity, and the permeability of water (Darcy through the foam was determined to be 180 in the direction of foam rise and 160 in the perpendicular to foam rise.

### Example 3

### Fabrication of a Cross-Linked Reticulated Polyurethane Matrix

A crosslinked Polyurethane Matrix was made using similar starting materials and following procedures similar to the one described in Example 1. The starting ingredients were same except for the following. The aromatic isocyanate Mondur MRS-20 (from Bayer AG) was used as the isocyanate component. Mondur MRS-20 (from Bayer AG), which is a liquid at 25° C., contains 4,4′-MDI and 2,4′-MDI and has an isocyanate functionality of about 2.3. Glycerol or Glycerin 99.7% USP/EP (from Dow Chemicals) was used as a cross-linker and 1,4-Butanediol (from BASF Chemical) was used as chain extender. The cross-linker and the chain extender are mixed into system B. The proportions of the components that were used are set forth in the following table:

TABLE 4

| Ingredient | Parts by Weight |
|---|---|
| PolyCD ™ CD220 (g) | 100 |
| Propylene carbonate (g) | 5.80 |
| Tegostab BF-2370 (g) | 1.50 |
| Ortegol 501 (g) | 2.00 |
| Mondur MRS-20 (g) | 51.32 |
| Isocyanate index | 1.0 |
| Distiled water) (g) | 1.89 |
| Glycerine (g) | 2.15 |
| Chain extender (g) | 0.72 |
| Dabco 33 LV (g) | 0.56 |

The reaction profile is as follows:

| | |
|---|---|
| Mixing time of System A and System B before | 10 |
| pouring into cardboard box (seconds) | |
| Cream time (seconds) | 27 |
| Rise time (seconds) | 120 |

Reticulation of the foam described above was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

A second reticulation was performed on the once reticulated elastomeric matrix structure using similar condition reticulation parameters as described above to yield a reticulated elastomeric matrix structure in which cell walls between adjoining pores were further removed.

A scanning electron micrograph image of the reticulated elastomeric matrix of this example (not shown here) demonstrated, e.g., the communication and interconnectivity of pores therein.

The average pore diameter of the twice reticulated elastomeric matrix, as determined from optical microscopy observations, was greater than about 222 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 4.3 lbs/ft³ (0.069 g/cc) was obtained.

Tensile tests of twice reticulated elastomeric matrix were conducted on samples that were cut perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm, and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 37.2 psi (26,500 kg/m²). The elongation to break perpendicular to the direction of foam rise was determined to be 89%. The average tensile strength parallel to the direction of foam rise was determined as 70.4 psi (49,280 kg/m²). The elongation to break perpendicular to the direction of foam rise was determined to be 109%.

Compressive tests of twice reticulated elastomeric matrix were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths parallel to the direction of foam rise at 50% and 75% compression strains were determined to be 3.3 psi (2,310 kg/m²) and 10.7 psi (7,490 kg/m²), respectively.

The compression set of twice reticulated elastomeric matrix, determined after subjecting the reticulated sample to 50% compression for 22 hours at 25° C. then releasing the compressive stress, parallel to the direction of foam rise, was determined to be about 5.1%.

The permeability of water (Darcy) through the twice reticulated elastomeric matrix was determined to be 226 in the direction of foam rise.

### Example 4

### Implant Assembly, Processing, and Testing

An experiment was performed to assess the pushability of different NEUROSTRING™ implant configurations based on various combinations of structural filaments. The material in Example 2 is used as the starting matrix for an implant according to the invention. Different combinations of structural filaments were used in the study, either multifilament fiber corresponding to fiber equivalent 7-0 polyester suture and/or platinum wire measuring 0.0015″-0.0030″ in diameter.

To create each implant prototype, a 2 mm sheet of the elastomeric matrix material was loaded into a commercial sewing machine. In the top bobbin, 7-0 multifilament fiber corresponding to 7-0 polyester suture available in a spool format (Genzyme) was used. In the bottom bobbin, a single platinum wire or a rope composite (7-0 multi-filament fiber corresponding to 7-0 polyester suture+platinum wire or platinum wire+platinum wire) was used as outlined in Table 5 below. The rope composites were created by using a coil winder to create a twisted composite of two filaments. After being sewn with the filaments, the elastomeric matrix was cut to the required implant length. The elastomeric matrix with the structural filaments was then trimmed under a microscope using surgical scissors (Fine Science Tools) to an outer diameter of 0.022″-0.023″. Platinum markers were then positioned over the length of the implant at 1.0 cm increments and crimped in place manually using tweezers.

The implants were attached to a pusher/detachment system. Each implant was pushed through a clear, custom-made 0.027″ ID microcatheter for a total of five passes to verify the pushability of the string. Pushability was selected as the most clinically relevant outcome measure which serves as a proxy for the stiffness or mechanical strength of the string. If the string was not pushable at the original length, the string was trimmed shorter to evaluate pushable length. The outcome of the pushability testing is set forth in the following table:

TABLE 5

| | Wire/Rope | Fiber | | | | | |
|---|---|---|---|---|---|---|---|
| Implant | Configuration | Type for | String | String | | | |
| Prototype | for Bottom | Top | Length | Dia. | MC ID | Pushable | |
| Number | Bobbin | Bobbin | (cm) | (inch) | (inch) | Y/N | Comments |
| 1 | 7-0 suture/ | 7-0 suture | 24 | .023 | .027 | Y | String starts to |
| | .003″ PT rope | | | | | | buckle after 3 |
| | | | | | | | passes through the |
| | | | | | | | microcatheter |
| 2 | .003″ Platinum | 7-0 suture | 22 | .018 | .027 | Y | No buckling at all |
| | Wire | equivalent | | | | | |
| 3 | .003″ Platinum | 7-0 suture | 18.5 | .026 | .027 | Y | Some buckling @ |
| | Wire | equivalent | | | | | 4^{th} pass |
| 4a | .002″ Platinum | 7-0 suture | 24 | .022 | .027 | N | |
| | Wire | equivalent | | | | | |
| 4b | .002″ Platinum | 7-0 suture | 20 | .022 | .027 | N | |
| | Wire | equivalent | | | | | |
| 4c | .002″ Platinum | 7-0 suture | 15 | .022 | .027 | N | |
| | Wire | equivalent | | | | | |
| 4d | .002″ Platinum | 7-0 suture | 10 | .022 | .027 | Y | Some buckling @ |
| | Wire | equivalent | | | | | 2^{nd} pass |
| 5 | 7-0 Suture | 7-0 suture | N/A | N/A | N/A | N | Ultra Soft string- |
| | | equivalent | | | | | not pushable |
| 6a | Secant Suture/ | 7-0 suture | 17 | .032 | .033 | N | |
| | .0015″ (2) PT | equivalent | | | | | |
| | Wire | | | | | | |
| 6b | Secant Suture/ | 7-0 suture | 10 | .032 | .033 | Y | Buckling @ 1^{st} |
| | .0015″ (2) PT | equivalent | | | | | pass |
| | Wire | | | | | | |
| 7a | 7-0 Suture/ | 7-0 suture | 22 | .023 | .027 | N | |
| | .002″ (2) PT | equivalent | | | | | |
| | Rope | | | | | | |
| 7b | 7-0 Suture/ | 7-0 suture | 15 | .023 | .027 | Y | Buckling @ 1^{st} |
| | .002″ (2) PT | equivalent | | | | | pass |
| | Rope | | | | | | |
| 7c | 7-0 Suture/ | 7-0 suture | 10 | .023 | .027 | Y | Trimmed shorter - |
| | .002″ (2) PT | equivalent | | | | | buckling @ 2^{nd} |
| | Rope | | | | | | pass |
| 8 | 7-0 suture/ | 7-0 suture | 18.5 | .024 | .027 | Y | No buckling at all |
| | .003″ (2)PT | equivalent | | | | | |
| | rope | | | | | | |
| 9a | 7-0 suture/ | 7-0 suture | 25 | .024 | .027 | N | |
| | .00225″ (2)PT | equivalent | | | | | |
| | rope | | | | | | |
| 9b | 7-0 suture/ | 7-0 suture | 15 | .024 | .027 | Y | Trimmed shorter - |
| | .00225″ (2)PT | equivalent | | | | | no buckling |
| | rope | | | | | | |

Different types of implants according to the invention employing different combinations of structural filaments can be manufactured, and they have different functionalities.

### Example 5

### Mechanical Performance of Implant

For select prototypes, an INSTRON Universal Testing Instrument was used to determine the tensile properties of the implant prototypes. The implants were made by a process similar to the one described in Example 4 but using matrix material made in accordance to Example 3 . For these tested prototypes, three samples of 3.8 cm length each were tested at a crosshead speed of 2.54 cm/min and with the gauge length set at 0.7 inch (1.8 cm). The results were as follows:

TABLE 6

| | | | Tensile | Stiffness or slope |
|---|---|---|---|---|
| String | Length | | load | of load vs |
| Configuration | of | Avg. Dia. | to failure | extension curve |
| (Wire/Suture) | implant | (inch) | (Newtons) | (Newtons/mm). |
| 0.002″ PT + fiber | 8 | 0.02 | 5.7 | 19.2 |
| equivalent to | | | | |
| 7-0 Polyester | | | | |
| 0.002″ (X 2) + fiber | 8 | 0.02 | 7.2 | 46.8 |
| equivalent to | | | | |
| 7-0 Polyester | | | | |

Clearly, the mechanical properties of the implant according to the invention can be varied or engineered by the type and number of the reinforcing filament or fiber.

### Example 6

### Histological Evaluation of a Plurality of Crosslinked Reticulated Polyurethane Matrix Implants in a Canine Carotid Bifurcation Aneurysm Model

An established animal model of cerebral aneurysms was used to evaluate the histologic outcomes of implanting a plurality of cylindrical implants machined from a block of cross-linked reticulated polyurethane matrix as described in Example 2. The three animals were sacrificed at the three-month timepoint to assess tissue response to the cross-linked reticulated polyurethane matrix.

One of two different implant configurations was used in this experiment. The first configuration was a cylindrical implant measuring 6 mm diameter×15 mm length. The second configuration was a segmented, cylindrical implant measuring 3 mm diameter×15 mm length. To machine the implants, a rotating die cutter was used to cut 3 mm and 6 mm diameter cylinders. The implants were then trimmed to 15 mm in length. Implant dimensions were tested for acceptability by use of calipers and visualization under a stereo-microscope, with acceptance of implants measuring ±5% of target dimensions.

An aneurysm was surgically created at the carotid arterial bifurcation of three dogs. This model simulates the hemodynamics of a human saccular aneurysm, which typically occurs at an arterial bifurcation. After one month, a second embolization procedure was performed in which a plurality of implants machined from a block of cross-linked reticulated polyurethane matrix was delivered into the aneurysm sac using a guide catheter. The 6×15 mm cylindrical implants were delivered using a commercially available 7 Fr Cordis Vista-Brite guide catheter. The 3×15 mm cylindrical implants were delivered using a commercially available 5 Fr Cordis Vista-Brite guide catheter. A loader apparatus was used to pull compress the implants from their expanded state into a compressed state for introduction through the hemostasis valve of the guide catheter. An obturator was then used to push the compressed implant from the proximal end of the guide catheter to the distal end, where the implant was deployed in a slow, controlled manner into the aneurysm sac.

A plurality of implants was used in each of the three dogs to achieve post-procedural angiographic occlusion as shown in Table 7 below. Platinum coil markers (0.003″ diameter) embedded in the central lumen of the implants allowed the implants to be readily visualized under standard fluoroscopy, to verify implant deployment, placement, and positioning.

TABLE 7

| | | | 6 × 15 | 3 × 15 |
|---|---|---|---|---|
| | Aneurysm | Aneurysm | mm | mm |
| | Dimensions | Volume | Implants | Implants |
| Dog # | (mm) | (mm³) | (n) | (n) |
| BMX-1 | 22.4 mm L × 10.1 mm W | 1884 mm³ | 2 | 5 |
| BMX-2 | 18.9 mm L × 8.8 mm W | 1207 mm³ | 4 | 9 |
| BMX-3 | 23 mm L × 11 mm W | 2295 mm³ | 12 | 0 |

At three months following the embolization procedure, the animals were sacrificed to assess tissue response to the cross-linked reticulated polyurethane matrix. For histology processing, samples were dehydrated in a graded series of ethanol and embedded in methylmethacrylate plastic. After polymerization, each aneurysm was bisected (sawn) longitudinally by the Exakt method and glued onto a holding block for sectioning using a rotary microtome at 5-6 microns. The sections were mounted on charged slides and stained with hematoxylin-eosin and Movat pentachrome stains. All sections were examined by light microscopy for the presence inflammation, healing response, calcification and integrity of the wall at the neck interface and surrounding aneurysm.

Gross observation indicated that the aneurysm sac was fully packed with no open spaces. There was nearly complete pannus growth on the luminal surface at the proximal neck interface with focal, luminal invagination (pocket).

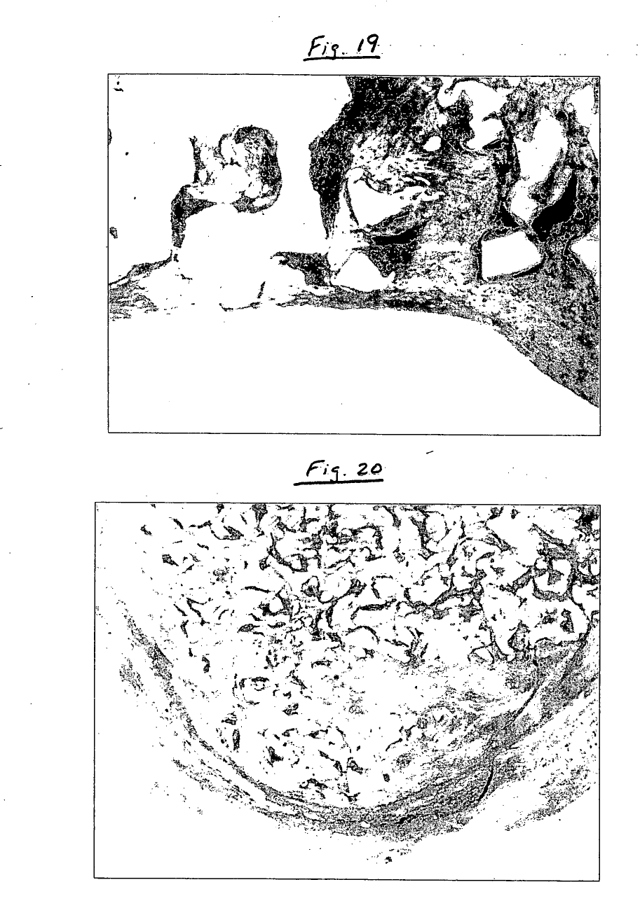

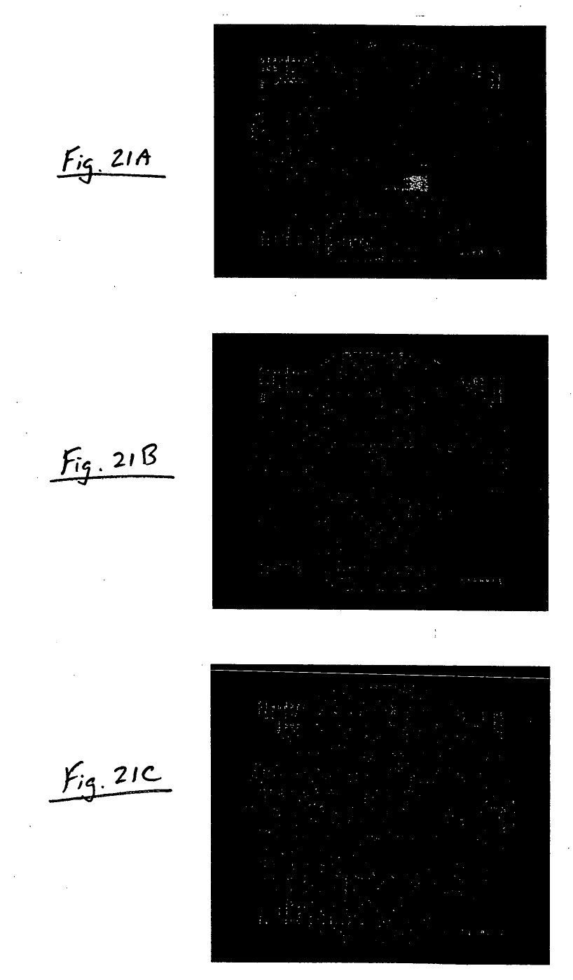

Longitudinal section through the proximal neck of the aneurysm showed greater than 95% luminal occlusion of aneurysm sac by reticulated polyurethane matrix. The luminal surface at the proximal interface showed almost complete covering by fibrous tissue with overlying endothelialization as shown in FIG. 19, which is 20× magnification showing fibrocollagenous tissue surrounding implant material and extending to luminal surface at proximal neck interface. There was nearly complete healing of tissue ingrowth surrounding the implanted material characterized by the presence of fibrocollagenous tissue (light-green and yellow by Movat Pentachrome stain) as shown in FIG. 20, which is a low power (4×) Movat stain of the apex of the aneurysm showing marked fibrocollagenous tissue ingrowth. There was minimal, focal organizing granulation tissue surrounding material (predominantly at the center of the occluded aneurysm) with mild, chronic inflammation consisting of lymphocytes and some giant cells, consistent with the healing response. There was almost complete replacement of elastic lamellae by fibrocollagenous tissue. No calcification was observed.

The histological response to the reticulated polyurethane matrix in this experiment demonstrated that the material can serve as a scaffold to support extensive organic tissue ingrowth with minimal inflammation and thereby holds promise as a bioactive solution to the treatment of cerebral aneurysms.

### Example 7

### Angiographic Outcomes from Use of Reticulated Polyurethane Implants in a Canine Carotid Bifurcation Aneurysm Model

An established animal model of cerebral aneurysms was used to evaluate the angiographic outcomes of implanting a 0.030″ implants according to the invention made from cross-linked reticulated polyurethane matrix as described in Example 2.

To create the implants, thin sheets measuring 2.0 mm in depth were sliced from a block of reticulated polyurethane matrix. A sewing machine was then used to stitch surgical suture measuring 0.003″ in diameter through the thin foam sheet to form a straight line. Individual strings were cut by using micro-scissors to trim around the suture line under a microscope until the final outer diameter of 0.030″ (outside edge of the foam string) was achieved. Implant dimensions were tested for acceptability by delivering each individual string through a custom-made 3.5 Fr (0.035″ inner diameter) microcatheter. Platinum bands were hand-crimped every 1.0 cm along the length of each implant to impart radiopacity.

An aneurysm was surgically created at the carotid arterial bifurcation of three dogs. This model simulates the hemodynamics of a human saccular aneurysm, which typically occurs at an arterial bifurcation. After one month, a second embolization procedure was performed as follows. After preparing the access site using standard surgical technique, a 6 Fr Boston Scientific Guide Catheter with Straight Tip was advanced to the aneurysm. A Boston Scientific Excelsior 3 Fr Microcatheter was then advanced through the guide catheter into the aneurysm neck. One or two GDC-18 framing coils were then deployed through the microcatheter to frame the aneurysm. After positioning and detaching the framing coil, the Excelsior microcatheter was withdrawn. A custom-made 3.5 Fr (0.035″ inner diameter) microcatheter was then advanced through the guide catheter into the aneurysm neck. The implant, loader, and pusher wire were removed from their sterile packaging. The loaded implant and microcatheter were flushed with sterile saline. The loader/implant was then introduced into the hemostasis valve of the microcatheter. The implant was subsequently delivered into the aneurysm by pushing the implant with the pusher wire while using hydraulic assistance through the 3.5 Fr custom microcatheter. The implant was positioned and detached into the aneurysm. The pusher wire was removed from the microcatheter and an angiogram was performed to confirm occlusion. Implants ranging from 10-18 cm in length were deployed as necessary until angiographic occlusion was confirmed.

Table 8 below shows the quantities and volumes of framing coils and implants used in each of the three animals. All 22 implants were successfully delivered using hydraulic assistance and controlled mechanical detachment. Post-procedure angiographic occlusion was achieved in all three animals, with minor neck remnants.

TABLE 8

| | | | Total | |
|---|---|---|---|---|
| | | | Implant | Number of |
| | Aneurysm | Framing | Length | Implant |
| Dog # | Dimensions | Coil Qty | (cm) | Implants |
| BMX-4 | 13.2 mm L × 12.1 mm W | 2 | 59.0 cm | 5 |
| BMX-5 | 14.0 mm L × 10.2 mm W | 1 | 100.5 cm | 8 |
| BMX-6 | 15.6 mm L × 10.2 mm W | 1 | 109.5 cm | 9 |

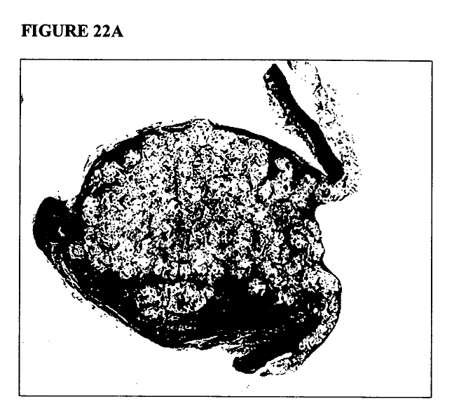

At two-week follow-up, an angiogram was performed to assess angiographic outcomes including device stability (compaction) and aneurysm recanalization. All three dogs showed stable or progressing occlusion with no device compaction and no evidence of aneurysm recanalization. The angiographic series from BMX-5 is shown in FIGS. 21A to 21B, where FIG. 21A represents pre-embolization, FIG. 21B represents post embolization, and FIG. 21C represents follow-up.

The angiographic outcomes at two-week follow-up demonstrated that implants according to the invention can be utilized for the embolization of cerebral aneurysms. This experiment showed the implant of the invention is consistently deliverable through a 3 Fr microcatheter, and that the implants are stable with no evidence of device compaction, no migration, and no aneurysm recanalization at the two-week followup timepoint.

### Example 8

### Effects of Packing Density on Angiographic Outcomes Using Reticulated Polyurethane Implants in a Canine Carotid Bifurcation Aneurysm Model

An established animal model of cerebral aneurysms was used to evaluate the impact of different packing densities on angiographic outcomes for two different configurations of implants machined from a block of cross-linked reticulated polyurethane matrix as described in Example 2. The study evaluated the efficacy of different packing densities using (i) cylindrical implants (3 mm×15 mm, 6 mm×15 mm) machined as described in Example 5; and (ii) 0.030″ implants machined as described in Example 7. Packing density effectiveness was measured as angiographic occlusion and device stability (no compaction) at two-week follow-up.

Table 9 below shows that packing densities ranging from 40% -350% result in angiographic occlusion at two-week follow-up with stable or progressing occlusion and no device compaction. The one exception, BMX-1, was noted to occur in a dog with an unusual, giant, unstable aneurysm that continued to expand even at the two-week angiographic follow-up timepoint.

TABLE 9

| | | | Embolization | |
|---|---|---|---|---|
| | | | Agents | |
| | Aneurysm | Packing | (Reticulated Matrix | 2W Angiographic |
| | Volume | Density | “RM” and/or GDC- | Outcomes vs. |
| Dog # | (mm³) | (%) | 18 Coils) | Baseline |
| PILOT | 1457.0 mm³ | 349.2% | 12-6 × 15 mm RM | 100% occlusion |
| | | | Cylinders | No recanalization |
| BMX-1 | 1907.8 mm³ | 166.7% | 6-6 × 15 mm RM | Recanalization |
| | | | Cylinders | |
| | | | 6-3 × 15 mm RM | |
| | | | Cylinders | |
| BMX-2 | 1196.3 mm³ | 115.2% | 2-6 × 15 mm RM | Progressing |
| | | | Cylinders | thrombosis |
| | | | 5-3 × 15 mm RM | No device |
| | | | Cylinders | compaction |
| BMX-3 | 766.3 mm³ | 345.8% | 4-6 × 15 mm RM | Stable occlusion |
| | | | Cylinders | No device |
| | | | 5-3 × 15 mm RM | compaction |
| | | | Cylinders | |
| BMX-4 | 1011.8 mm³ | 39.6% | 2-GDC-18 coils | No recanalization |
| | | | 59.0 cm RM | No device |
| | | | Implant | compaction |
| BMX-5 | 762.6 mm³ | 78.7% | 1-GDC-18 coil | Progressive |
| | | | 100.5 cm RM | occlusion |
| | | | Implant | No device |
| | | | | compaction |
| BMX-6 | 849.7 mm³ | 76.6% | 1-GDC-18 coil | Progressive |
| | | | 109.5 cm RM | occlusion |
| | | | Implant | No device |
| | | | | compaction |

This experiment demonstrated that various configurations of implants machined from reticulated polyurethane matrix can be utilized to embolize large aneurysms in a wide range of packing densities (40% -350%) with efficacious angiographic outcomes at two-week foollow-up.

### Example 9

### Histological Evaluation of 0.030″ Diameter Implants in a Canine Carotid Bifurcation Aneurysm Model

An established animal model of cerebral aneurysms was used to evaluate the angiographic outcomes of implanting 0.030″ NEUROSTRING™ implants according to the invention made from cross-linked reticulated polyurethane matrix as described in Example 2.

The implants were prepared as described in Example 7. Thin sheets measuring 2.0 mm in depth were sliced from a block of reticulated polyurethane matrix. A sewing machine was then used to stitch 7-0 surgical polyester suture with 0.003″ diameter through the thin foam sheet to form a straight line. Individual strings were cut by using micro-scissors to trim around the suture line under a microscope until the final outer diameter of 0.030″ (outside edge of the foam string) was achieved. Implant dimensions were tested for acceptability by delivering each individual string through a custom-made 3.5 Fr (0.035″ inner diameter) microcatheter. Platinum bands (90% Pt, 10% Ir, 0.016 in. i.d., 0.002 in. wall, 0.030 in. length) were hand-crimped every 1.0 cm along the length of each implant to impart radiopacity.

The animal model and surgical procedure were also performed as described in Example 7. An aneurysm was surgically created at the carotid arterial bifurcation of three dogs. This model simulates the hemodynamics of a human saccular aneurysm, which typically occurs at an arterial bifurcation. After one month, a second embolization procedure was performed as follows: After preparation of the access site using standard surgical technique, a 6 Fr Boston Scientific Guide Catheter with Straight Tip was advanced to the aneurysm. A Boston Scientific Excelsior 3 Fr Microcatheter was then advanced through the guide catheter into the aneurysm neck. A single GDC-18 framing coil measuring either 14 mm×30 cm (BMX-5) or 16 mm×30 cm (BMX-6) was then deployed through the microcatheter to frame the aneurysm. After positioning and detaching of the framing coil, the Excelsior microcatheter was withdrawn. A custom-made 3.5 Fr (0.035″ inner diameter) microcatheter was then advanced through the guide catheter into the aneurysm neck. The implant, loader, and pusher wire were removed from their sterile packaging. The loaded implant and microcatheter were flushed with sterile saline. The loader/implant was then introduced into the hemostasis valve of the microcatheter. The implant was subsequently delivered into the aneurysm by pushing the implant with the pusher wire while using hydraulic assistance through the 3.5 Fr custom microcatheter. The implant was positioned and detached into the aneurysm. The pusher wire was removed from the microcatheter and an angiogram was performed to confirm occlusion. Implants ranging from about 10 to 18 cm in length were deployed as necessary until angiographic occlusion was confirmed:

The table below outlines the aneurysm dimensions, test article utilization and packing density in this study:

TABLE 10

| | | | Total | Embolization | | |
|---|---|---|---|---|---|---|
| | | Aneurysm | Length of | Agent | | Packing |
| | Aneurysm | Volume | Implant | Volume | Number of | Density |
| Dog # | Dimensions | (mm³) | (cm) | (mm³) | Implants | (%) |
| BMX-5 | 14.0 mm L × 10.2 mm | 762.6 mm³ | 100.5 cm | 600.0 mm³ | 8 | 78.7% |
| | W × 6.4 mm | | | | | |
| | Neck | | | | | |
| BMX-6 | 15.6 mm L × 10.2 mm | 849.7 mm³ | 109.5 cm | 650.7 mm³ | 9 | 76.6% |
| | W × 6.7 mm | | | | | |
| | Neck | | | | | |

Both animals were sacrificed at the three-month timepoint for histological evaluation. For histology processing, samples were dehydrated in a graded series of ethanol and embedded in methylmethacrylate plastic. After polymerization, each aneurysm was bisected (sawn) longitudinally by the Exakt Method. The Exakt sections were glued onto plastic slides for grinding and polishing to 25-30 microns thickness and stained with Toluidine Blue. All sections were examined by light microscopy for the presence inflammation, healing response, calcification and integrity of the wall at the neck interface and surrounding aneurysm.

Grossly, there was nearly complete pannus growth on the luminal surface at the proximal neck interface indicating good sealing of the neck interface. A longitudinal section through the proximal neck of the aneurysm showed total occlusion of the aneurysm sac by the Neurostring implant and surrounding coil framing. The luminal surface at the neck interface showed complete covering by neointima formation with overlying endothelialization. The periphery of the sac showed thin to thick tissue healing (in-growth) with minimal to mild chronic inflammation (normal healing response) surrounding the implants. The inner two thirds of the aneurysm sac was densely packed with the embolic material and surrounded by minimal to mild organizing granulation tissue with fibrin deposition. There was minimal to mild chronic inflammation consisting of predominantly of macrophages and some giant cells.

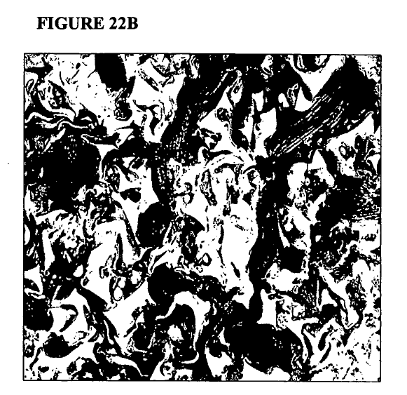

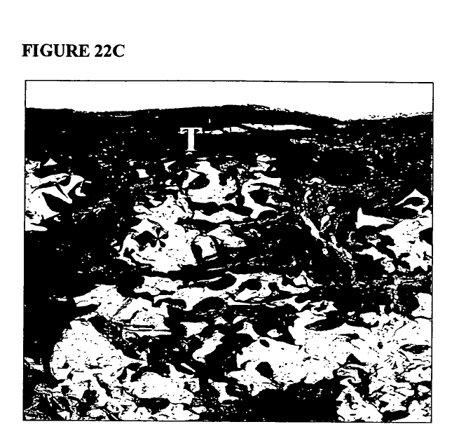

The effectiveness of the implant according to the invention can be appreciated in FIGS. 22A to 22C. FIG. 22A is a low power (1.25 magnification)(TB stained) micrograph of a longitudinal (Exakt) section through the proximal neck of the aneurysm showing complete neointimal coverage of neck surface and total occlusion of aneurysm sac by embolic material in subject BMX-5. FIG. 22B is a high power micrograph of a representative section showing the center (core) of the aneurysm for the same subject with progressive healing characterized by granulation tissue (pink staining) with fibrin surrounding the embolic material (void spaces). FIG. 22C is a high power micrograph showing thin to thick tissue ingrowth (T) at the periphery of the aneursym for the subject BMX-6 and surrounding underlying embolic implant material (*).

Thus, there was effective occlusion in the canine carotid bifurcation aneurysm model of aneurysms treated with NEUROSTRING™ implants by the three-month timepoint. Each of the treated aneurysms showed complete healing (neointima formation) and confluent endothelialization of the neck surface with adequate incorporation of organizing granulation tissue surrounding the embolic material at the periphery of the sac. In both samples, the center of the sac showed adequate distribution of embolic material characterized by minimal to partial organization and fibrin deposition, indicative of the healing process underway. Overall, both samples show minimal to mild chronic inflammation, which is associated with the normal tissue healing response.

While illustrative embodiments of the invention have been described, it is, of course, understood that various modifications of the invention will be obvious to those of ordinary skill in the art. Such modifications are within the spirit and scope of the invention which is limited and defined only by the appended claims.

We claim:

1. A vascular occlusion device comprising:
a flexible, longitudinally extending biocompatible member, and
at least one longitudinally extending component coupled to the biocompatible member at various points to secure the biocompatible member and assist it in conformally filling a targeted vascular site.

2. The device of claim 1 which assumes a non-linear shape to conformally fill a targeted vascular site.

3. The device of claim 1 which comprises a non-curvilinear shape in at least one portion of the member.

4. The device of claim 3, wherein the non-curvilinear shape comprises at least one vertex.

5. The device of claim 4, wherein the at least one vertex comprises a plurality of vertices.

6. The device of claim 5, wherein the plurality of vertices permit chain-like folding of the device.

7. The device of claim 1, wherein the biocompatible member comprises an elastomeric matrix.

8. The device of claim 7, wherein the elastomeric matrix is a biodurable, reticulated elastomeric matrix.

9. The device of claim 7, wherein the elastomeric matrix is selected from the group consisting of polycarbonate polyurethane-urea, polycarbonate polyurea-urethane, polycarbonate polyurethane, and polycarbonate polysiloxane polyurethane.

10. The device of claim 7, wherein the elastomeric matrix comprises resiliently recoverable material.

11. The device of claim 1, wherein each longitudinally extending component comprises a structural filament.

12. The device of claim 1, wherein the at least one longitudinally extending components comprise a polymeric fiber or filament and at least one wire element.

13. The device of claim 12, wherein the at least one wire element comprises a continuous wire.

14. The device of claim 12, wherein the at least one wire element comprises a plurality of staples.

15. The device of claim 14, wherein the plurality of staples are interlocked pairwise to form a chain.

16. The device of claim 1 which comprises at least two longitudinally extending components that are coupled to each other at a plurality of locations.

17. The device of claim 16, wherein the components are coupled by knotting.

18. The device of claim 1, wherein the at least one longitudinally extending components comprise at least two structural filaments.

19. The device of claim 18, wherein there are two structural filaments.

20. The device of claim 18, wherein the structural filaments are selected from materials preselected to vary at least one physical property of the device.

21. The device of claim 20, wherein the physical property is stiffness.

22. The device of claim 20, wherein the physical property comprises modulus of elasticity.

23. The device of claim 18, wherein each structural filament is selected from the group consisting of platinum wire, polymeric fiber or filament, a braid of platinum wire and polymeric fiber or filament, and a braid of two or more platinum wires.

24. The device of claim 18, where the structural filaments are knotted together by radiopaque bands.

25. The device of claim 1, wherein at least one longitudinally extending component comprises radiopaque material.

26. The device of claim 16, wherein the material of each component and the coupling between the at least two components are selected to produce a desired physical property of the device.

27. The device of claim 26, wherein the desired physical property of the device comprises a stiffness in at least one portion of the device.

28. The device of claim 27, wherein the stiffness in at least one portion of the device comprises a stiffness at a location of coupling and the stiffness comprises a stiffness relative to a stiffness of the device at a point substantially distant from the point of coupling.

29. The device of claim 1 which is capable of occluding an aneurysm.

30. The device of claim 29, wherein the aneurysm is a cerebral aneurysm.

31. The device of claim 1 which is capable of occluding a vessel.

32. The device of claim 1 which is capable of occluding a vascular malformation.

33. An introducer system for a vascular occlusion device, the vascular occlusion device having a proximal end and a distal end, the distal end having a contact element, the system comprising:
an introducer component having a longitudinally extending lumen and proximal and distal ends;
a pusher component slidable within the introducer component, the pusher component having a distal end positioned adjacent to the distal end of the occlusion device; and
a core component having a distal end and extending through the pusher component and parallel to the occlusion device so that the distal end of the core component contacts the contact element, thereby applying a tensile force to the occlusion device.

34. The system of claim 33, further comprising:
an interlocking wire having a distal end extending longitudinally into the pusher member,
wherein:
the occlusion device has a release element at its proximal end,
the distal end of the pusher component has an opening through which the release element extends,
the distal end of the interlocking wire is releasably held within the distal end of the pusher member, and
the distal end of the interlocking wire releasably engages the release element so that the distal end of the pusher component releasably engages the proximal end of the occlusion device.

35. The system of claim 33, wherein the release element comprises a loop.

36. The system of claim 33, wherein the contact element comprises a tensioning element.

37. A method for occluding a targeted vascular site comprising:
introducing an introducer system into a delivery catheter having a longitudinally extending lumen and proximal and distal ends, the introducer system carrying a vascular occlusion device and having a pusher component;
withdrawing the introducer system, leaving the vascular occlusion device positioned within the lumen of the delivery catheter;
advancing the vascular occlusion device using the pusher component to position the vascular occlusion device within the targeted vascular site;
disengaging the pusher component from the occlusion device; and
withdrawing the pusher.

38. A device for occluding a targeted vascular site comprising:
an elongate occluding element comprising a material permitting ingrowth of tissue at the targeted vascular site; and
a plurality of features provided along the occluding element at preselected locations, the features selected to confer material characteristics allowing the creation of vertices in the element.

39. The device according to claim 38, wherein the vertices facilitate packing of the occluding element into the targeted vascular site.

40. The device according to claim 38, wherein at least one of the features comprises a topological characteristic of the elongate element.

41. The device according to claim 38, further comprising a second element coupled to the elongate element, wherein at least one of the features comprises a topological characteristic of the second element.

42. The device according to claim 41, further comprising a third element coupled to the elongate element, wherein at least one of the features comprises a relationship between the second and third elements.

43. The device according to claim 41, wherein the elongate element comprises a biodurable material permitting vascular tissue ingrowth and the second element comprises a polymeric fiber or filament.

44. The device according to claim 43, wherein the topological characteristic of the polymeric fiber or filament comprises a stitch.

45. The device according to claim 42, wherein the relationship between the second and third elements comprises a knot.

46. The device according to claim 38, wherein at least one of the group consisting of a dimension of a feature and a distance between a pair of features is preselected to facilitate packing of the targeted vascular site.

47. A method for treating a condition at a targeted vascular site comprising the steps of:
providing an elongate occlusion device comprising biocompatible material;
introducing the occlusion device into the targeted vascular site; and
while introducing the occlusion device, inducing at least one noncurvilinear geometry in the occlusion device.

48. The method of claim 47, wherein the step of inducing at least one non-curvilinear geometry produces a geometry of the occlusion device that packs the targeted vascular site in a substantially conformal manner.

49. The method of claim 47, wherein the at least one non-curvilinear geometry comprises a plurality of folds.

50. The method of claim 49, wherein the step of inducing a plurality of folds produces a chain-like occlusion device for packing the targeted vascular site in a substantially conformal manner.

51. The method of claim 47, wherein the occlusion device comprises a biocompatible material.

52. The method of claim 51, wherein the biocompatible material comprises a material permitting ingrowth of tissue at the targeted site.

53. The method of claim 52, wherein the occlusion device is introduced to permanently biointegrate at the targeted site.

54. A method for treating an aneurysm in a mammal, comprising the steps of:
providing an elongate biocompatible, biodurable material permitting tissue ingrowth at the site of the aneurysm; and
introducing the biocompatible, biodurable material at the site of the aneurysm in a quantity sufficient to occlude the aneurysm and to permit permanent biointegration of the occlusion device in the aneurysm.

55. The method of claim 54, wherein the biocompatible, biodurable material is a reticulated elastomeric matrix.

56. A method for treating an aneurysm comprising the step of introducing sufficient biocompatible material into the aneurysm to pack the aneurysm with the material to a packing density of from at least about 10% to at least about 200%.

57. The method of claim 56, wherein the biocompatible material comprises a flexible, longitudinally extending biocompatible member.

58. The method of claim 56, wherein the aneurysm is a cerebral aneurysm.

59. The method of claim 56, wherein the biocompatible material comprises non-swellable material.

60. A mechanism for detaching a vascular implant from a delivery device, the vascular implant having a proximal end and a coupling component at its proximal end, the mechanism comprising:
an engagement element coupled at a distal end of the delivery device, the engagement element having a first, engaged position and a second, disengaged position; and
an energy transfer component coupled to the engagement element at a distal portion of the component to actuate the engagement element;
wherein the engagement element, when actuated, engages the coupling component of the implant when in the first position and releases the coupling component when in the second position.

61. The mechanism of claim 60, wherein the coupling component of the implant comprises a flexible structure.

62. The mechanism of claim 61, wherein the flexible structure comprises at least one opening through which an aspect of the engagement element of the delivery device may pass when in the first, engaged position.

63. The mechanism of claim 62, wherein the flexible structure comprises a loop.

64. The mechanism of claim 60, wherein the engagement element comprises a structure that moves, along an axis, from the first position to the second position.

65. The mechanism of claim 64, wherein the delivery device comprises at least one of the group consisting of a wire and a sheath, the axis is parallel to the longitudinal axis of the delivery device, and the energy transfer component comprises at least one of the wire and the sheath.

66. The mechanism of claim 65, wherein the delivery device comprises a sheath and the energy transfer component comprises a wire, and wherein the engagement element transitions between the first position and the second position as a result of a relative rotation of the wire engagement element with respect to the delivery device sheath.

67. The mechanism of claim 66, wherein the engagement element comprises a distal portion of the wire, the coupling component of the implant comprises a loop structure, and wherein, in the first position of the engagement element, the loop structure is stably retained about a distal portion of the wire and, wherein, in the second position of the engagement element, the loop structure is released over a free distal end of the wire.

68. The mechanism of claim 67, wherein:
the distal portion of the wire has threads that engage mating threads coupled to the sheath,
the delivery device comprises a distal portion having a side wall with an aperture through which the loop structure passes and is held in place when the engagement element is in the first position, and
when the engagement element is in the second position, the distal end of the wire is proximal of the aperture, releasing the loop structure and allowing it to exit through the aperture.

69. The mechanism of claim 60, wherein the control element is operable by a practitioner.

70. A method for fabricating a vascular occlusion device, comprising the steps of:
providing a biocompatible material adapted for tissue ingrowth and capable of being formed into at least one elongate element having a longitudinal axis and dimensioned for vascular insertion;
coupling at least one support element to the biocompatible material to at least partially lie substantially along at least a portion of the longitudinal axis of the at least one elongate element; and
forming the elongate element from the biocompatible material substantially in the vicinity of the longitudinal axis.

71. The method of claim 70, wherein the elongate element comprises a flexible linear element.

72. The method of claim 71, wherein the at least one support element comprises a structural filament coupled to the biocompatible material substantially along at least a portion of its longitudinal axis.

73. The method of claim 72, wherein the at least one support element comprises a polymeric fiber or filament.

74. The method of claim 73, wherein the polymeric fiber or filament is stitched to the biocompatible material.

75. The method of claim 73, wherein the polymeric fiber or filament is coupled to the biocompatible material with at least one adhesive.

76. The method of claim 74, wherein the stitching is performed by a sewing machine.

77. The method of claim 73, wherein the at least one support element further comprises a second support element.

78. The method of claim 77, wherein the second support element comprises a staple.

79. The method of claim 77, wherein the at least one support element comprises at least two staples interlocking with one another form a chain.

80. The method of claim 77, wherein the at least one second support element comprises a radiopaque material.

81. The method of claim 77, wherein the at least one second support element comprises wire.

82. The method of claim 81, wherein the wire is coupled to the suture at a plurality of points.

83. The method of claim 82, wherein the coupling at at least one of the plurality of points comprises a knot.

84. The method of claim 77, wherein the at least one support element comprises at least two elements including a braided platinum wire/polymeric fiber or filament subassembly and a polymeric fiber or filament element.

85. The method of claim 77, wherein the at least second support element comprises a plurality of staples.

86. The method of claim 85, wherein the staples are spaced apart from one another.

87. The method of claim 70, wherein the step of forming the elongate element from the biocompatible material and the coupled support element comprises separating the elongate element and the support element from adjoining material.

88. The method of claim 87, wherein the step of separating is accomplished by cutting.

89. The method of claim 88, comprising the further step of removing excess material so that the elongate element has a preselected maximum width.

90. The method of claim 70, further comprising the step of coupling a visualizable element proximate to the end of the elongate element.

91. The method of claim 90, wherein the visualizable end unit comprises a coil.

92. The method of claim 90, wherein the end unit comprises radiopaque material.

93. The method of claim 70, wherein the length of the elongate element is from about 1 mm to about 1500 mm.

94. The method of claim 93, wherein the length of the elongate element is from about 50 mm to about 250 mm.

95. The method of claim 70, wherein the width of the elongate member is about 0.25 mm to about 12 mm.

96. The method of claim 95, wherein the width is about 0.25 mm to about 0.5 mm.

97. The method of claim 93, wherein the biocompatible material comprises an elastomeric matrix sheet material having a thickness of from about 1 mm to about 2 mm.

98. The method of claim 74, wherein the stitching of the suture to the biocompatible material forms a continuous stitch line from about 100 mm to about 500 mm long.

99. The method of claim 70, wherein the step of coupling at least one support element to the biocompatible material precedes the step of forming the elongate element from the biocompatible material, whereby the elongate element so formed includes the at least one support element.

100. The method of claim 70, wherein the step of forming the elongate element from the biocompatible material precedes the step of coupling at least one support element to the biocompatible material.

101. A method of treating an aneurysm comprising the steps of:
providing a biocompatible element having a form that comprises no predefined geometry; and
introducing the biocompatible element to conformally fill the aneurysm.

102. The method of claim 101, wherein the step of introducing the biocompatible material comprises application of the material to a wall of the aneurysm in such a manner that material curves upon itself to produce segments of the material.

103. The method of claim 102, wherein the material segments so applied are arranged in a brush stroke form.

104. The method of claim 102, wherein the segments, although substantially parallel to the wall of the aneurysm, each have a spatial orientation, and the spatial orientations of the segments are substantially randomly distributed with respect to one another.

105. The method of claim 102, wherein the segments are defined in situ by vertices in the material.

106. The method of claim 102, wherein the segments are defined by curved portions of the material that lack vertices.

107. The method of claim 101, wherein the step of introducing the material to conformally fill the aneurysm comprises application of a first layer of the material directly adjacent a wall of the aneurysm and a second layer substantially overlaying the first layer.

108. The method of claim 107, further comprising steps of applying additional layers until the aneurysm is substantially occluded.

109. The method of claim 101, the step of introducing the biocompatible element to fill the aneurysm comprises the deposition of the material in the manner of a viscous liquid flow.

110. The method of claim 101, wherein the material has a stiffness preselected to produce, when the material is fully introduced into the aneurysm, a packing density of from at least about 10% to at least about 200%.

111. The method of claim 101, wherein the step of introducing the biocompatible material to fill the aneurysm comprises the deposition of the material in the manner of a piece of cooked spaghetti to form a string ball in the aneurysm.

112. A vascular occlusion device comprising a string-shaped biocompatible element having a plurality of concavities for accommodating ingrowth of vascular tissue.

113. The vascular occlusion device of claim 112, wherein the concavities comprise pores.

114. The vascular occlusion device of claim 112, wherein the concavities together form a honeycomb structure.

115. The vascular occlusion device of claim 112, wherein the concavities together form a reticulated porous structure.

116. The vascular occlusion device of claim 112, wherein the concavities comprise a plurality of fragmentary pores.

117. The vascular occlusion device of claim 112, substantially excluding complete pores.

118. The vascular occlusion device of claim 112, wherein the concavities comprise cavities.

119. The vascular occlusion device of claim 112, wherein the concavities comprise concave surfaces formed in the exterior surface of the member.

120. The vascular occlusion device of claim 112, wherein, when the member is packed into an aneurysm, concavities are positioned adjacent one another and at least some of the adjacent concavities in neighboring portions of the member together form virtual pores to accommodate tissue ingrowth.

121. The vascular occlusion device of claim 112, wherein the average largest transverse dimension of the concavities is at least about 50 μm.

122. The vascular occlusion device of claim 121, wherein the average largest transverse dimension of concavities is at least about 100 μm.

123. The vascular occlusion device of claim 122, wherein the average largest transverse dimension of concavities is at least about 150 μm.

124. The vascular occlusion device of claim 112, wherein the average largest transverse dimension of concavities is at least about 200 μm.

125. The vascular occlusion device of claim 112, wherein the average largest transverse dimension of concavities is at least about 250 μm.

126. The vascular occlusion device of claim 112, wherein the average largest transverse dimension of concavities greater than about 250 μm.

127. The vascular occlusion device of claim 112, wherein the average largest transverse dimension of concavities is at least about 275 μm.

128. The vascular occlusion device of claim 112, wherein the average largest transverse dimension of concavities is at least about 300 μm.

129. The vascular occlusion device of claim 112, wherein the average largest transverse dimension of concavities is greater than about 300 μm.

130. The vascular occlusion device of claim 112, wherein the average largest transverse dimension of concavities is not greater than about 500 μm.

131. The vascular occlusion device of claim 112, wherein the average largest transverse dimension of the concavities is from about 200 to about 500 microns.

132. A vascular occlusion device comprising:
a flexible, longitudinally extending biocompatible member for delivery through a lumen of a delivery device;
the member comprising a plurality of pores having a dimensional characteristic selected on the basis of a minimum interior dimension of the lumen.

133. The vascular occlusion device of claim 132, wherein the interior dimension of the lumen comprises the inner diameter of the lumen, and the member has a maximum width less than the minimum interior dimension of the lumen.

134. The vascular occlusion device of claim 133, wherein the pore size is selected in order that the average pore diameter is greater than or equal to about 25% of the maximum width of the member.

135. The vascular occlusion device of claim 134, wherein the pore size is selected in order that the average pore diameter is from about 25% to about 33% of the maximum diameter of the member.

136. A system for adjusting the properties of a longitudinally extending device, comprising:
(a) a flexible, longitudinally extending member and
(b) at least one longitudinally extending component coupled to member (a) at various points,
wherein component (b) is selected from materials preselected to vary at least one physical property of the device.

137. The system of claim 136, wherein member (a) is biocompatible.

138. The system of claim 136, wherein component (b) is selected from the group consisting of platinum, iridium, and polymeric fibers or filaments.

139. The system of claim 136, wherein there are at least two longitudinally extending components.
